(19) United States
(12) Patent Application Publication
(10) Pub. No.: US 2006/0172925 A1
(43) Pub. Date: Aug. 3, 2006

(54) Thio-siRNA aptamers

(75) Inventors: David G. Gorenstein, Houston, TX (US); Xianbin Yang, Webster, TX (US)

Correspondence Address: CHALKER FLORES, LLP, 2711 LBJ FRWY, Suite 1036, DALLAS, TX, 75234, US

(73) Assignee: Board Of Regents, The University Of Texas System, Austin, TX (US)

(21) Appl. No.: 10/968,574

(22) Filed: Oct. 19, 2004

Related U.S. Application Data
(63) Continuation-in-part of application No. 10/758,488, filed on Jan. 15, 2004.
Application No. 10/758,488 is a continuation-in-part of application No. 10/272,509, filed on Oct. 16, 2002.
Application No. 10/272,509 is a continuation-in-part of application No. 09/425,804, filed on Oct. 25, 1999, now Pat. No. 6,867,289.
Application No. 09/425,804 is a division of application No. 09/425,798, filed on Oct. 25, 1999, now Pat. No. 6,423,493.
(60) Provisional application No. 60/105,600, filed on Oct. 26, 1998.

(51) Int. Cl. A61K 48/00; C07H 21/02

(52) U.S. Cl. 514/8; 536/23.1; 530/352; 514/44

(57) ABSTRACT

The present invention includes thioaptamers that are partially thio-modified, methods and compositions for the isolation, selection, improvement, characterization and use of RNA and DNA thioaptamers for gene silencing, including degradative and non-degradative interference with translation.

### CROSS-REFERENCE TO RELATED APPLICATIONS

This application is a continuation-in-part and claims priority based on U.S. patent application Ser. No. 10/758,488, filed Jan. 15, 2004, which is a continuation in part of U.S. patent application Ser. No. 10/272,509, filed Oct. 16, 2002, which is a continuation of U.S. patent application Ser. No. 09/425,804, filed Oct. 25, 1999, which is a divisional of U.S. patent application Ser. No. 09/425,798, filed Oct. 25, 1999, now U.S. Pat. No. 6,423,493, which claims priority to U.S. Provisional Application Ser. No. 60/105,600, filed Oct. 26, 1998.

The U.S. Government may own certain rights in this invention pursuant to the terms of the DARPA (9624-107 FP) and NIH (A127744).

### TECHNICAL FIELD OF THE INVENTION

The present invention relates in general to the field of thioaptamers, and more particularly, to thioaptamers for drug discovery, evaluation and characterization of physiological pathways that silence or interfere with gene expression.

### BACKGROUND OF THE INVENTION

Without limiting the scope of the invention, its background is described in connection with oligonucleotide agents that interfere with mRNA translation. RNA interference (RNAi) is one type of gene silencing in which duplex RNA, either endogenous to cells or delivered exogenous to the cells, interferes with the function of an exogenous or an endogenous gene through a complex form of hybridization to and cleavage of a target mRNA transcript. In the first report of RNAi, in studies on C. elegans, it was noted that: (1) interference was observed only for a double-stranded RNA (dsRNA) (not for single-stranded RNA (ssRNA)) sequence within the region of homology of the target gene; (2) that only a few molecules of dsRNA were required per affected cell, arguing against stoichiometric interference with endogenous mRNA and suggesting an amplification component in the interference mechanism; (3) that dsRNA segments corresponding to intron and promoter sequences do not produce interference (which is consistent with a post-transcriptional mechanism of gene silencing); and (4) dsRNA produces a pronounced decrease or elimination of the endogenous mRNA transcript (Fire, et al., 1998).

It is now known that RNAi is a manifestation of a broader group of post-transcriptional RNA silencing phenomena common to most eukaryotes that can be used to suppress expression of virtually any gene. RNAi, also called “RNA silencing,” reflects an elaborate cellular apparatus that eliminates abundant but defective mRNAs and defends against molecular parasites such as transposons and viruses. Indeed, the main physiological function of RNAi is assumed to be defense against viral infections (Gitlin 2002). Transcription of the silenced gene is unperturbed, but the mRNA transcript for the gene fails to accumulate to its normal cytoplasmic level. Thus, the gene is copied to mRNA in the nucleus, but the mRNA is destroyed, probably in cytoplasm, as soon as it is made.

Research in gene silencing in plants demonstrated that the silenced plants always contained small RNAs of about 25 nucleotides in length, derived from the sequence of the silenced gene. Such small RNAs are never found in plants that do not display silencing. The small RNAs include both sense and anti-sense fragments of the silenced gene. Similar small RNAs are found in extracts of insect cells pretreated with dsRNA. These “small interfering RNAs” are double-stranded and they are chopped from longer dsRNA by an ATP-dependent ribonuclease called “Dicer.”

Work in mammalian cell culture using dsRNA of between 38-1662 bp has failed to induce specific RNA interference. Indeed, long double-stranded nucleic acids, such as poly IC, have been known to induce the innate immune response (interferon inducer), whereas shorter double-stranded nucleic acids less than 25 nucleotides (nt) apparently do not induce interferon. However, 21-23 nt dsRNA (siRNAs) having overhanging 3′ ends, do mediate sequence-specific mRNA degradation in cultured mammalian cells (Elbashir 2001a, McCaffrey 2002, Caplen 2001). Thus, in humans, siRNAs are 21-23 nt dsRNA generally bearing two-nucleotide 3′overhanging ends. Synthetic siRNAs with the structure of the Dicer products are now routinely used to trigger gene silencing in cultured human cells.

Recent studies have shown siDNA analogs of anti-glucose-6-phosphate dehydrogenase siRNA had somewhat lower silencing activity and similar duration of activity, that RNA:DNA hybrid analogs had both enormously greater silencing activity and duration relative to siRNA. Christian claims that the RNA:DNA hybrids can not only silence genes with greater specificity than siRNAs, but can be introduced into cells without transfection, and may be effective against bacterial genes (J. S. Lamberton and A. T. Christian, Molecular Biotechnology, 24(2), 111-120 (2003)—Varying the nucleic acid composition of siRNA molecules dramatically varies the duration and degree of gene silencing).

What is needed are methods and compositions that permit for the rapid detection, isolation and evaluation of siDNA oligonucleotides and double stranded thioaptamer including RNA:DNA hybrids that have reduced susceptibility to nucleases, that are sequence specific, have an activity that is equal to, or modified from, the activity of a wild-type siRNA or that has one or more activities that are not available for a wild-type RNAi molecule.

### SUMMARY OF THE INVENTION

The present invention permits the rapid detection, isolation and evaluation of small RNA oligonucleotides that have reduced susceptibility to nucleases, that are sequence specific, have a gene silencing activity that is equal to, or modified from, the activity of, e.g., a wild-type small, interfering RNA (siRNA); a micro, interfering RNA (mRNA); a small, temporal RNA (stRNA); short, hairpin RNA (shRNA); small, interfering DNA (siDNA); or even a short, hairpin DNA (shDNA). The compositions and methods of the present invention include “thio-modified nucleotide aptamers” or “thioaptamers” that specifically bind to a target molecule or portion thereof and mediate gene silencing. The effects of thioaptamer binding may be detected at a variety of levels and using a variety of read-outs as disclosed herein and as known in the growing art of RNA interference. Generally, modulation of the functional attributes of bioactive targets is achieved initially by specific thioaptamer binding followed by interference with translation or degradation of a target. Binding may, for example, interrupt protein-DNA, protein-RNA, RNA-DNA, RNA-RNA and/or DNA-DNA interactions such as those that occur between a DICER complex and RNA in the modification of gene expression.

The present invention includes an isolated thioaptamer that mediates gene silencing. The thioaptamer may include, e.g., a terminal 3′ hydroxyl group and include ribonucleotides or deoxyribonucleotides. The portion of the thioaptamer that is modified may include one or more of the following, rATP(αS), rUTP(αS), rGTP(αS), rCTP(αS), rATP(αS₂), rUTP(αS₂), rGTP(αS₂) or rCTP(αS₂), alone or in combination. In another embodiment, the portion of the thioaptamer that is modified may include one or more of the following, dATP(αS), dTTP(αS), dGTP(αS), dCTP(αS), dATP(αS₂), dTTP(αS₂), dGTP(αS₂) or dCTP(αS₂), alone or in combination. The thioaptamer may be made using a method in which a polymerase, e.g., a DNA, an RNA polymerase or even a reverse transcriptase is used to incorporate the dNTP'S or rNTP's with thiophosphate substitutions so that the thioaptamer has monothioate or dithioate substitutions. Generally, the thioaptamer will be from about 21 to about 25 nucleotides in length, however, modification of the thioaptamer intracellularly may decrease or increase the length of actual active gene silencing thioaptamers. The thioaptamers of the present invention may be, e.g., a double stranded thioaptamer with a perfect complementarity match to a target gene wherein gene silencing occurs by mRNA cleavage; a thioaptamer with an imperfect complementarity match to a target gene wherein gene silencing occurs by repressed translation of mRNA to protein; or a single-stranded thioaptamer with perfect complementarity match to a target gene wherein gene silencing occurs by mRNA cleavage. Additionally, the thioaptamer may be a double stranded thioaptamer including: RNA:DNA, RNA:RNA, RNA:PNA and DNA:PNA hybrids. The thioaptamer may be a portion of a RNA-induced silencing complex (RISC) complex and/or produced by a DICER complex.

In one embodiment, the thioaptamer may be, e.g., a short interfering RNA (siRNA); a micro, interfering RNA (mRNA); a small, temporal RNA (stRNA); or a short, hairpin RNA (shRNA). In some cases, the thioaptamer provided to a cell or cellular extract may be a thioaptamer precursor, e.g., a long dsRNA or an about 70 nucleotide stem-loop RNA (shRNA). Mature thioaptamers will generally be a double stranded thioaptamer of about 21 to about 25 nucleotides long or a single-stranded thioaptamer that is about 15 to about 22 nucleotides long or even up to about 28 nucleotides long. In one embodiment, gene silencing may be by degradation of an mRNA transcript that is cleaved in the presence of the thioaptamer before it can express a protein. Alternatively, gene silencing may be accomplished by the regulation of translation when the thioaptamer binds an mRNA transcript at or about its 3′UTR.

In another embodiment, the thioaptamer may be, e.g., a short interfering DNA (siDNA); a micro, interfering DNA (miDNA); a small, temporal DNA (stDNA); or a short, hairpin DNA (shDNA). In some cases, the thioaptamer provided to a cell or cellular extract may be a thioaptamer precursor. Mature thioaptamers will generally be a double stranded thioaptamer of about 21 to about 25 nucleotides long or a single-stranded thioaptamer that is about 15 to about 22 nucleotides long or even up to about 28 nucleotides long. In one embodiment, gene silencing may be by degradation of an mRNA transcript that is cleaved in the presence of the thioaptamer before it can express a protein. Alternatively, gene silencing may be accomplished by the regulation of translation when the thioaptamer binds an mRNA transcript at or about its 3′UTR.

The present invention also include a method of producing a mature thioaptamer of from about 21 to about 23 nucleotides in length that includes the steps of, combining a double-stranded precursor thioaptamer with a soluble extract that mediates gene silencing, thereby producing a precursor-extract mixture; and maintaining the precursor-extract mixture under conditions in which the double-stranded thioaptamer is processed to the mature thioaptamer of from about 21 to about 23 nucleotides in length. The method may also include isolating the thioaptamer of from about 21 to about 23 nucleotides from the precursor-extract mixture. The method may also include the step of determining the sequence of the mature thioaptamer and the location of one or more thio-modifications to the mature thioaptamer. Upon isolation, selection or improvement of selective binding the method may further include the steps of, determining the sequence of the mature thioaptamer and the location of one or more thio-modifications to the mature thioaptamer; and chemically synthesizing the mature thioaptamer, e.g., a mature thioaptamer of about 21 to about 23 nucleotides that is produced by the method disclosed herein.

Another method of the present invention is mediating gene silencing of a target gene in a cell or organism by introducing a thioaptamer of from about 21 to about 23 nucleotides in length into the cell or organism and maintaining the cell or organism under conditions in which gene silencing occurs, thereby mediating expression of the target gene in the cell or organism. In one example, the thioaptamer may be optimized for RNase H degradation and thereby cause gene silencing. Examples of target genes include: endogenous and exogenous genes (e.g., viral or cellular genes), transgenes and the like. The compositions and methods of the present invention may be used to make a knockdown cell or organism to, e.g., mimic a disease. Target cells may include cells in any stage of development, e.g., stem cells. Using the thioaptamers disclosed herein, the function of a gene may be examined in a cell or organism by introducing a thioaptamer of from about 21 to about 23 nucleotides that targets an mRNA of the gene for gene silencing into the cell or organism, thereby producing a test cell or test organism; maintaining the test cell or test organism under conditions under which gene silencing of mRNA of the gene occurs, thereby producing a test cell or test organism in which mRNA of the gene is silenced and observing the phenotype of the test cell or test organism against an appropriate control cell or control organism to provide information about the function of the gene.

The present invention also includes a method of assessing whether a gene product is a suitable target for drug discovery by introducing an RNA thioaptamer that mediates gene silencing of from about 21 to about 25 nucleotides into a cell or organism under conditions in which gene silencing of an mRNA for the target gene results in decreased expression of the gene; and determining the effect of the decreased expression of the gene on the cell or organism, wherein if decreased expression has an effect, then the gene product is a target for drug discovery. In one embodiment, the thioaptamer may be part of a pharmaceutical composition, e.g., a thioaptamer of about 21 to about 25 nucleotides that mediates thioaptamer gene silencing and an appropriate carrier.

The thioaptamers of the present invention may also be used as part of a method of identifying target sites within an mRNA that are efficiently targeted for gene silencing by combining an RNA thioaptamer corresponding to a sequence of a labeled mRNA to be degraded under conditions in which labeled mRNA is degraded. Next, the sites in the mRNA that are efficiently cleaved are identified. The RNA thioaptamer may be part of a a thioaptamer library, e.g., a pool of thioaptamers from a thioaptamer library or even a library of libraries. In an alternative method, target sites may be identified within an mRNA that are efficiently targeted for gene silencing by combining an RNA thioaptamer corresponding to a sequence of a labeled mRNA under conditions in which labeled mRNA is not degraded and the protein level is reduced.

The present invention also includes a combinatorial thioaptamer library that includes two or more unique thioaptamers that include a combination of backbone modifications and sequence that mediates gene silencing of an mRNA to which it corresponds. The thioaptamers may be attached covalently to one or more beads, e.g., polystyrene/polydivinyl benzene copolymer. The thioaptamers may include one or more phosphorothioate linkages, one or more phosphorodithioate linkages and/or one or more methylphosphonate linkages. The thioaptamer may include, e.g., a viral sequence, a genomic sequence and/or an expressed sequence. The thioaptamers may also include a detectable agent, e.g., a colorimetric, a fluorescent, a radioactive and/or an enzymatic agent. The thioaptamers disclosed herein may also include a strand complementary to the thioaptamer. The library of thioaptamers may be, e.g., created by a split and pool combinatorial synthesis chemistry.

One example of a library is a one-bead, one-thioaptamer combinatorial library that includes, two or more beads, wherein attached to each bead is a unique thioaptamer comprising a single unique sequence, wherein each unique thioaptamer includes a unique mix of modified and unmodified nucleotides and wherein the thioaptamer mediates gene silencing of an mRNA to which it corresponds. Alternatively, the one-bead, one-thioaptamer combinatorial library may be two or more beads, wherein attached to each bead is a unique thioaptamer comprising an imperfect complementarity match to a target gene to form a thioaptamer-bead, wherein each unique thioaptamer-bead comprises a unique mix of modified and unmodified nucleotides and wherein the thioaptamer mediates gene silencing of an mRNA to which it has imperfect complementarity. In yet another example, the combinatorial library is a bead library of thioaptamer libraries, wherein each bead comprises a thioaptamer library of imperfect complementarity to a target sequence for gene silencing.

Using the RNA thioaptamers disclosed herein it is possible to reduce the expression of a gene in a cell by selecting a thioaptamer that mediates gene silencing of the gene to which it corresponds and introducing the thioaptamer into the cell, wherein the thioaptamer mediates RNA interference of a targeted sequence. Sequences that may be targeted by the RNA thioaptamers of the present invention include, e.g., gene markers, splice acceptors, splice donors, IRES, recombinase sites, promoters, ori sequences, cloning sites, and intervening sequence. Target cells include non-mammalian, plant, yeast, bacterial, mammalian, human and even stem cells. The thioaptamer may be an antisense molecule and may even be a ribozyme.

In one use, the RNA thioaptamers may be used to attenuate expression of a target gene in cultured cells, by introducing an RNA thioaptamer into the cells in an amount sufficient to attenuate expression of the target gene, wherein the RNA thioaptamer includes a nucleotide sequence that hybridizes under stringent conditions to a nucleotide sequence of the target gene and mediates attenuation of protein expression for a gene to which it corresponds. The method may further include the step of activating a gene silencing activity in the cell.

In one example, RNAi may be used as a potential alternative to transgenic mice, where the knock-out effect could be turned on and off. Potential limitations in using dsRNA to knock-out a gene function may include: (1) a sequence shared between closely related genes might interfere with several members of the gene family; (2) a low level of expression might resist RNAi for some or all genes; and/or (3) a small number of cells might escape RNAi so that one does not get complete loss of function as one would get with a knockout mouse (Fire, et al., 1998).

The present invention may be used to control, study, evaluate or even diagnose a biological pathway using the thioaptamers of the present invention by using the thioaptamer in a method that uses some of the steps below, depending on the nature of the host cell. For example, mammals use steps 2-4, below. In contrast, plants and worms use steps 1-4 in which dsRNA is amplified by RdRPs.
Step 1—dsRNA is amplified by RNA-dependent RNA polymerases (RdRPs);
Step 2—dsRNA is chopped up by Dicer to 21-23 nt siRNAs;
Step 3—the siRNA is incorporated into an RNA-induced silencing complex (RISC) containing an endonuclease, with the siRNA then guiding the endonuclease to the site of its complementary sequence on the mRNA, and the RISC proceeds to cut up the mRNA at that site, destroying the mRNA; and
Step 4—the siRNA produced by Dicer also acts as a primer in amplifying dsRNA, which is then acted upon by Dicer, producing more siRNA (Step 2). RISC activity is generally believed to be restricted to the cytoplasm.

Control of gene expression with nucleic acids such as short antisense oligonucleotides (ss DNA targeting homologous complementary mRNA) is a powerful tool for investigating protein function inside cells (Koller, 2000) and may provide a major new class of therapeutics (Jansen and Zangemeister-Wittke, 2002; Opalinska and Gewirtz, 2002; Braasch and Corey, 2002). It has been has pointed out that dsRNAs represent a potential addition to therapeutic nucleic acid control of gene expression (Zamore, 2002). Steps 1 and 2 in the above mechanism can be bypassed by transfection of chemically synthesized 21-23 nt dsRNAs, called small interfering RNA or siRNAs. Knowing only the DNA sequence of a gene, one could design sequence-specific siRNA to inhibit expression of that gene. By turning off the mRNA of that gene, one could study the function of that gene. Thus, RNAi has potential both as a therapeutic and as a tool for the study of physiological pathways.

In vitro studies in human cell culture have demonstrated siRNA inhibition of retroviral infection utilizing delivery of exogenous synthetic siRNA against HIV challenge (Novina, 2002; Jacque, 2002; Lee, 2002), RSV challenge (Hu, 2002) and HCV challenge (Yokota, et al., 2003) and transfection with a plasmid expressing siRNA targeting poliovirus (Gitlin, 2002) and hepatitis C virus (Yokota, et al., 2003). It has also been demonstrated, in mouse models, that siRNAs can function in vivo, inhibiting gene expression of both endogenous genes (Wianny 2000, Xia 2002, McCaffrey 2002, Song 2003) and exogenous genes (McCaffrey 2003). The latter report demonstrated siRNA inhibition of hepatitis B virus in mice. It has also been shown in mammalian cell models that siRNA can be used to target disease-causing mutant alleles (e.g., single-nucleotide polymorphisms (SNPs)) of genes suggesting therapeutic application to SNP-linked diseases (Miller, et al., 2003).

It has been demonstrated that siRNA-mediated gene silencing is sufficiently specific and reliable to allow large-scale screening of gene function and drug target validation via gene expression profiling following siRNA delivery (Semizarov, et al., 2003). In another study, microarray gene expression studies in siRNA gene silencing did not indicate detectable off-target gene silencing, as had previously been reported in C. elegans work (Chi, et al., 2003). In another study using Dharmacon-supplied siRNAs, however, off-target silencing was observed (Jackson, et al., 2003), which may have been due to excess siRNA in the cell, indicating that siRNA levels must be optimized (RNAi Roundup article on GenomeWeb internet site).

A study comparing siRNA to antisense oligonucleotides for gene knockdown indicated that in terms of dose-response, siRNA had an IC₅₀ that was 100-fold lower than that of antisense oligonucleotides, and that as is the case for antisense, siRNA efficacy differed at different target sites on the mRNA target (Miyagishi, et al., 2003). It is difficult to predict a priori the most effective target site for a siRNA design, however, it was observed that siRNAs generated in vitro by recombinant human Dicer typically have high RNAi activity (Kawasaki, et al., 2003), offering an optimization path.

One embodiment of the present invention includes an isolated thioaptamer that mediates gene silencing, wherein the thioaptamer is a double-stranded hybrid thioaptamer. Another embodiment of the present invention that mediates gene silencing, includes a thioaptamer comprises a combination of short interfering DNA (siDNA); a micro, interfering DNA (miDNA); a small, temporal DNA (stDNA); or a short, hairpin DNA (shDNA). The thioaptamer may include, e.g., a terminal 3′ hydroxyl group and include ribonucleotides or deoxyribonucleotides. The thioaptamer may be made by incorporating dNTP's or rNTP's with thiophosphate substitutions so that the thioaptamer has monothioate or dithioate substitutions are formed. One embodiment of the modified protion of the thioaptamer may include one or more of the following: rATP(αS), rUTP(αS), rGTP(αS), rCTP(αS), rATP(αS2), rUTP(αS2), rGTP(αS2), rCTP(αS2), dATP(αS), dTTP(αS), dGTP(αS), dCTP(αS), dATP(αS2), dTTP(αS2), dGTP(αS2), or dTTP(αS2) alone or in combination. The thioaptamer may be made chemically or enzymatically using polymerase, e.g., a DNA, an RNA polymerase or a reverse transcriptase.

One embodiment of the present invention includes a double-stranded hybrid thioaptamer having a perfect complementarity match or an imperfect complementarity match to a target gene and gene silencing occurs by mRNA cleavage. Other embodiments may include a double-stranded hybrid thioaptamer having an perfect complementarity match or an imperfect complementarity match to a target gene and gene silencing occurs by repressed translation of mRNA to protein.

One embodiment of the present invention is a method of mediating gene silencing of a target gene in a cell or organism including the steps of: introducing a double-stranded hybrid thioaptamer into the cell or organism; and maintaining the cell or organism under conditions in which gene silencing occurs, thereby mediating expression of the target gene in the cell or organism. The target gene may encode a viral gene or a cellular gene.

Another embodiment of the present invention is a method of examining the function of a gene in a cell or organism including the steps of, introducing a double-stranded hybrid thioaptamer that targets an mRNA of the gene for gene silencing into the cell or organism, thereby producing a test cell or test organism; maintaining the test cell or test organism under conditions under which gene silencing of mRNA of the gene occurs, thereby producing a test cell or test organism in which mRNA of the gene is silenced; and observing the phenotype of the test cell or test organism against an appropriate control cell or control organism to provide information about the function of the gene.

Yet another embodiment of the present invention is a method of assessing whether a gene product is a suitable target for drug discovery including the steps of, introducing an double-stranded hybrid thioaptamer that mediates gene silencing into a cell or organism under conditions in which gene silencing of an mRNA for the target gene results in decreased expression of the gene; and determining the effect of the decreased expression of the gene on the cell or organism, wherein if decreased expression has an effect, then the gene product is a target for drug discovery. Another embodiment of the present invention is a pharmaceutical composition having a double-stranded hybrid thioaptamer that mediates thioaptamer gene silencing and an appropriate carrier.

Still other embodiments of the present invention includes a method for reducing the expression of a gene in a cell, having the steps of: selecting a double-stranded hybrid thioaptamer that mediates gene silencing of the gene to which it corresponds; and introducing the thioaptamer into the cell, wherein the thioaptamer mediates RNA interference of a targeted sequence.

One embodiment of the present invention includes a method for attenuating expression of a target gene in cultured cells, having the step of, introducing a double-stranded hybrid thioaptamer into the cells in an amount sufficient to attenuate expression of the target gene, wherein the double-stranded hybrid thioaptamer comprises a nucleotide sequence that hybridizes under stringent conditions to a nucleotide sequence of the target gene and mediates attenuation of protein expression for a gene to which it corresponds. In other embodiments the thioaptamer may including a combination of short interfering DNA (siDNA); a micro, interfering DNA (miDNA); a small, temporal DNA (stDNA); or a short, hairpin DNA (shDNA). The cell may be in cell culture, infected with a virus, a human cell, a mammalian cell or a stem cell.

Another embodiment includes a method of producing a double-stranded hybrid thioaptamer comprising the steps of, combining a double-stranded hybrid thioaptamer precursor with a soluble extract that mediates gene silencing, thereby producing a precursor-extract mixture; and maintaining the precursor-extract mixture under conditions in which the double-stranded hybrid thioaptamer is processed to the mature thioaptamer. Other embodiments may include the additional steps of isolating the double-stranded hybrid thioaptamer from the precursor-extract mixture, determining the sequence of the double-stranded hybrid thioaptamer and the location of one or more thio-modifications to the thioaptamer; and chemically synthesizing the thioaptamer.

One embodiment of the present invention includes a method of mediating gene silencing of a target gene in a cell or organism having the steps of, introducing a thioaptamer into the cell or organism, wherein the thioaptamer includes a combination of short interfering DNA (siDNA); a micro, interfering DNA (miDNA); a small, temporal DNA (stDNA); or a short, hairpin DNA (shDNA); and maintaining the cell or organism under conditions in which gene silencing occurs, thereby mediating expression of the target gene in the cell or organism. The target gene may encode a viral gene or cellular gene. The method of gene silencing may be further defined as degradation of an mRNA transcript of the target gene that is cleaved in the presence of the thioaptamer before it can express a protein or by the regulation of translation of the target gene when the thioaptamer binds an mRNA transcript of the target gene at or about its 3′UTR.

Another embodiment of the present invention includes a combination of short interfering DNA (siDNA); a micro, interfering DNA (miDNA); a small, temporal DNA (stDNA); or a short, hairpin DNA (shDNA) having a perfect complementarity match or an imperfect complementarity match to a target gene and gene silencing occurs by mRNA cleavage. Other embodiments may include a combination of short interfering DNA (siDNA); a micro, interfering DNA (miDNA); a small, temporal DNA (stDNA); or a short, hairpin DNA (shDNA) having an perfect complementarity match or an imperfect complementarity match to a target gene and gene silencing occurs by repressed translation of mRNA to protein. The thioaptamer may be a portion of a RNA-induced silencing complex (RISC) complex and/or produced by a DICER complex.

One embodiment, includes a method of examining the function of a gene in a cell or organism including the steps of: introducing a thioaptamer comprises a combination of short interfering DNA (siDNA); a micro, interfering DNA (miDNA); a small, temporal DNA (stDNA); or a short, hairpin DNA (shDNA) that targets an mRNA of the gene for gene silencing into the cell or organism, thereby producing a test cell or test organism; maintaining the test cell or test organism under conditions under which gene silencing of mRNA of the gene occurs, thereby producing a test cell or test organism in which mRNA of the gene is silenced; and observing the phenotype of the test cell or test organism against an appropriate control cell or control organism to provide information about the function of the gene.

Yet another embodiment of the present invention includes a method of assessing whether a gene product is a suitable target for drug discovery including the steps of, introducing a thioaptamer comprises a combination of short interfering DNA (siDNA); a micro, interfering DNA (miDNA); a small, temporal DNA (stDNA); or a short, hairpin DNA (shDNA) that mediates gene silencing into a cell or organism under conditions in which gene silencing of an mRNA for the target gene results in decreased expression of the gene; and determining the effect of the decreased expression of the gene on the cell or organism, wherein if decreased expression has an effect, then the gene product is a target for drug discovery.

Still other embodiments of the present invention include pharmaceutical compositions having a thioaptamer that mediates thioaptamer gene silencing and an appropriate carrier, wherein the thioaptamer including a combination of short interfering DNA (siDNA); a micro, interfering DNA (miDNA); a small, temporal DNA (stDNA); or a short, hairpin DNA (shDNA).

Another embodiment of the present invention includes a method for reducing the expression of a gene in a cell, having the steps of, selecting a thioaptamer comprises a combination of short interfering DNA (siDNA); a micro, interfering DNA (miDNA); a small, temporal DNA (stDNA); or a short, hairpin DNA (shDNA) that mediates gene silencing of the gene to which it corresponds; and introducing the thioaptamer into the cell, wherein the thioaptamer mediates RNA interference of a targeted sequence.

Still another embodiment of the present invention includes a method of producing a thioaptamer having the steps of: combining a combination of short interfering DNA (siDNA); a micro, interfering DNA (miDNA); a small, temporal DNA (stDNA); or a short, hairpin DNA (shDNA) thioaptamer precursor with a soluble extract that mediates gene silencing, thereby producing a precursor-extract mixture; and maintaining the precursor-extract mixture under conditions in which a combination of short interfering DNA (siDNA); a micro, interfering DNA (miDNA); a small, temporal DNA (stDNA); or a short, hairpin DNA (shDNA) thioaptamer is processed to the mature thioaptamer. Other embodiment may include the steps of isolating the thioaptamer from the precursor-extract mixture or determining the sequence of the thioaptamer and the location of one or more thio-modifications to the mature thioaptamer. Yet other embodiments may include thioaptamer produced by the method.

### BRIEF DESCRIPTION OF THE DRAWINGS

For a more complete understanding of the features and advantages of the present invention, reference is now made to the detailed description of the invention along with the accompanying figures in which:

FIG. 1 is a gel that shows the titration of RNA aptamers and a VEE Capsid protein;

FIGS. 2A, 2B and 2C are stem-loop structures for three aptamers including a variant of the 16_{—}1 aptamer, the 7-7 aptamer and a third related aptamer, respectively;

FIG. 3 is a gel of the titration of the RNA aptamer 16_{—}1 with the VEE Capsid Protein;

FIG. 4 is a graph that demonstrates the specificity of the 16_{—}1 RNA aptamer;

FIG. 5 summarizes the selection modification cycle used to prepare RNA thioaptamers that combine sequence specificity and thio-modification to the aptamers;

FIGS. 6A, 6B, 6C and 6D are stem-loop structures for three engineered RNA aptamers derived from 16_{—}1;

FIG. 7 is a graph that shows the effect of thio-modification of the aptamer on siRNA gene silencing in HeLa cells;

FIG. 8 is a graph that shows the effect of thio-modification of the aptamer on siRNA gene silencing in HeLa cells;

FIG. 9 is a graph that shows the effect of thioaptamers on siRNA gene silencing in HeLa cells;

FIG. 10 is another graph that shows the effect of thioaptamers on siRNA gene silencing in HeLa cells;

FIG. 11 is a graph that shows the silencing by thioaptamers in HeLa cells;

FIG. 12 is a graph that shows the silencing by native siRNAs on luciferase gene silencing in HeLa cells;

FIG. 13 is a graph that shows the silencing by thiophosphate siRNAs on luciferase gene silencing in HeLa cells;

FIG. 14 is a graph that shows luciferase activity because of thiophosphate siRNAs generated in HeLa cells;

FIGS. 15A and 15B are graphs that show the effects of thiophosphate siRNAs on HeLa cells cytotoxicity; and

FIG. 16 is a graph of the effects of thiophosphate siRNAs on luciferase gene silencing in HeLa cells.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a,” “an” and “the” are not intended to refer to only a singular entity, but include the general class of which a specific example may be used for illustration. The terminology herein is used to describe specific embodiments of the invention, but their usage does not delimit the invention, except as outlined in the claims.

As used herein, the term “hydrid thioaptamer,” “hybrid thio-modified aptamer,” are used to describe are used interchangeably to describe oligonucleotides (ODNs) (or libraries of thioaptamers) in which one or more of the four constituent nucleotide bases of an oligonucleotide are analogues or esters of nucleotides that normally form the DNA or RNA backbones and wherein such modification confers increased nuclease resistance; and the DNA or RNA may be single or double stranded. e.g., RNA:DNA, RNA:RNA, RNA:PNA and DNA:PNA hybrids in which the “hydrid thioaptamer,” “hybrid thio-modified aptamer” may be used as transcription factor decoys, short interfering DNA (siDNA); micro, interfering DNA (miDNA); small, temporal DNA (stDNA); short, hairpin DNA (shDNA); short interfering RNA (siRNA); micro, interfering RNA (mRNA); small, temporal RNA (stRNA); or short, hairpin RNA (shRNA) and the like, that mediate gene silencing into a cell or organism under conditions in which gene silencing of an mRNA for the target gene results in decreased expression of the gene. For example, the hydrid thioaptamer can include one or more monophosphorothioate or phosphordithioate linkages selected by incorporation of modified backbone phosphates through polymerases from wherein the group: dATP(αS), dTTP(αS), dCTP(αS), dGTP(αS), rUTP(αS), rATP(αS), rCTP(αS), rGTP(αS), dATP(αS₂), dTTP(αS₂), dCTP(αS₂), dGTP(αS₂), rATP(αS2), rCTP(αS₂), rGTP(αS₂) and rUTP(αS₂) or modifications or mixtures thereof. Phosphoromonothioate or phosphorodithioate linkages may also be incorporated by chemical synthesis or by DNA or RNA synthesis by a polymerase, e.g., a DNA or an RNA polymerase or even a reverse transcriptase, or even thermostable or other mutant versions thereof. In another example, no more than three adjacent phosphate sites of the modified nucleotide aptamer are replaced with phosphorothioate groups. In yet another example, at least a portion of non-adjacent dA, dC, dG, dU, dT, rA, rC, rG, rT or rU) phosphate sites of the hydrid thioaptamer are replaced with phosphorothioate groups. In another example of a hydrid thioaptamer, all of the non-adjacent dA, dC, dG, dU, dT, rA, rC, rG, rT or rU) phosphate sites of the hydrid thioaptamer are replaced with phosphorothioate groups; all of the non-adjacent dA, dC, dG, dU, dT, rA, rC, rG, rT or rU) phosphate sites of the hydrid thioaptamer are replaced with phosphorothioate groups; or substantially all non-adjacent phosphate sites of the hydrid thioaptamer are replaced with phosphorothioate groups. In still another embodiment of the present invention, no more than three adjacent phosphate sites of the hydrid thioaptamer are replaced with phosphorodithioate groups. The thioaptamers may be obtained by adding bases enzymatically using a mix of four nucleotides, wherein one or more of the nucleotides are a mix of unmodified and thiophosphate-modified nucleotides, to form a partially thiophosphate-modified thioaptamer library. In another example of “hydrid thioaptamer” these are made by adding bases to an oligonucleotide wherein a portion of the phosphate groups are thiophosphate-modified nucleotides, and where no more than three of the four different nucleotides are substituted on the 5′-phosphate positions by 5′-thiophosphates in each synthesized oligonucleotide are thiophosphate-modified nucleotides. In still another embodiment of the present invention, peptide nucleic acids (PNAs) may be incorporated.

As used herein, “synthesizing” of a random combinatorial library refers to chemical methods known in the art of generating a desired sequence of nucleotides including where the desired sequence is random. Typically in the art, such sequences are produced in automated DNA synthesizers programmed to the desired sequence. Such programming can include combinations of defined sequences and random nucleotides.

“Random combinatorial oligonucleotide library” means a large number of oligonucleotides of different sequence where the insertion of a given base at given place in the sequence is random. “PCR primer nucleotide sequence” refers to a defined sequence of nucleotides forming an oligonucleotide which is used to anneal to a homologous or closely related sequence in order form the double strand required to initiate elongation using a polymerase enzyme. “Amplifying” means duplicating a sequence one or more times. Relative to a library, amplifying refers to en masse duplication of at least a majority of individual members of the library.

As used herein, “thiophosphate” or “phosphorothioate” are used interchangeably to refer to analogues of DNA or RNA having sulphur in place of one or more of the non bridging oxygens bound to the phosphorus. Monothiophosphates or phosphoromonothioates [αS] have only one sulfur and are thus chiral around the phosphorus center. Dithiophosphates are substituted at both oxygens and are thus achiral. Phosphoromonothioate nucleotides are commercially available or can be synthesized by several different methods known in the art. Chemistry for synthesis of the phosphorodithioates has been developed by one of the present inventors as set forth in U.S. Pat. No. 5,218,088 (issued to Gorenstein, D. G. and Farschtschi, N., Jun. 8, 1993 for a Process for Preparing Dithiophosphate Oligonucleotide Analogs via Nucleoside Thiophosphoramidite Intermediates), relevant portions incorporated herein by reference.

As used herein, the terms “thio-modified aptamer” and “thioaptamer” are used interchangeably to describe oligonucleotides (ODNs) (or libraries of thioaptamers) in which one or more of the four constituent nucleotide bases of an oligonucleotide are analogues or esters of nucleotides that normally form the DNA or RNA backbones and wherein such modification confers increased nuclease resistance; and the DNA or RNA may be single or double stranded. For example, the modified nucleotide thioaptamer can include one or more monophosphorothioate or phosphordithioate linkages selected by incorporation of modified backbone phosphates through polymerases from wherein the group: dATP(αS), dTTP(αS), dCTP(αS), dGTP(αS), rUTP(αS), rATP(αS), rCTP(αS), rGTP(αS), dATP(αS₂), dTTP(αS₂), dCTP(αS₂), dGTP(αS₂), rATP(αS₂), rCTP(αS₂), rGTP(αS₂) and rUTP(αS₂) or modifications or mixtures thereof. Phosphoromonothioate or phosphorodithioate linkages may also be incorporated by chemical synthesis or by DNA or RNA synthesis by a polymerase, e.g., a DNA or an RNA polymerase or even a reverse transcriptase, or even thermostable or other mutant versions thereof. In another example, no more than three adjacent phosphate sites of the modified nucleotide aptamer are replaced with phosphorothioate groups. In yet another example, at least a portion of non-adjacent dA, dC, dG, dU, dT, rA, rC, rG, rT or rU) phosphate sites of the modified nucleotide aptamer are replaced with phosphorothioate groups. In another example of a thioaptamer, all of the non-adjacent dA, dC, dG, dU, dT, rA, rC, rG, rT or rU) phosphate sites of the modified nucleotide aptamer are replaced with phosphorothioate groups; all of the non-adjacent dA, dC, dG, dU, dT, rA, rC, rG, rT or rU) phosphate sites of the modified nucleotide aptamer are replaced with phosphorothioate groups; or substantially all non-adjacent phosphate sites of the modified nucleotide aptamer are replaced with phosphorothioate groups. In still another embodiment of the present invention, no more than three adjacent phosphate sites of the modified nucleotide aptamer are replaced with phosphorodithioate groups. The thioaptamers may be obtained by adding bases enzymatically using a mix of four nucleotides, wherein one or more of the nucleotides are a mix of unmodified and thiophosphate-modified nucleotides, to form a partially thiophosphate-modified thioaptamer library. In another example of “thioaptamers” these are made by adding bases to an oligonucleotide wherein a portion of the phosphate groups are thiophosphate-modified nucleotides, and where no more than three of the four different nucleotides are substituted on the 5′-phosphate positions by 5′-thiophosphates in each synthesized oligonucleotide are thiophosphate-modified nucleotides.

Thiophosphate nucleotides are an example of modified nucleotides. “Phosphodiester oligonucleotide” means a chemically normal (unmodified) RNA or DNA oligonucleotide. Amplifying “enzymatically” refers to duplication of the oligonucleotide using a nucleotide polymerase enzyme such as DNA or RNA polymerase. Where amplification employs repetitive cycles of duplication such as using the “polymerase chain reaction,” the polymerase may be, e.g., a heat stable polymerase, e.g., of Thermus aquaticus or other such polymerases, whether heat stable or not.

“Contacting” in the context of target selection means incubating a oligonucleotide library with target molecules. “Target molecule” means any molecule to which specific aptamer selection is desired. “Essentially homologous” means containing at least either the identified sequence or the identified sequence with one nucleotide substitution. “Isolating” in the context of target selection means separation of oligonucleotide/target complexes, preferably DNA/protein complexes, under conditions in which weak binding oligonucleotides are eliminated.

By “split synthesis” it is meant that each unique member of the combinatorial library is attached to a separate support bead on a two (or more) column DNA synthesizer, a different thiophosphoramidite or phosphoramidite is first added onto both identical supports (at the appropriate sequence position) on each column. After the normal cycle of oxidation (or sulfurization) and blocking (which introduces the phosphate, monothiophosphate or dithiophosphate linkage at this position), the support beads are removed from the columns, mixed together and the mixture reintroduced into both columns. Synthesis may proceed with further iterations of mixing or with distinct nucleotide addition.

Aptamers may be defined as nucleic acid molecules that have been selected from random or unmodified oligonucleotides (“ODN”) libraries by their ability to bind to specific targets or “ligands.” In one embodiment, an iterative process of in vitro selection may be used to enrich the library for species with high affinity to the target. The iterative process involves repetitive cycles of incubation of the library with a desired target, separation of free oligonucleotides from those bound to the target and amplification of the bound ODN subset using the polymerase chain reaction (“PCR”). The penultimate result is a sub-population of sequences having high affinity for the target. The sub-population may then be subcloned to sample and preserve the selected DNA sequences. These “lead compounds” are studied in further detail to elucidate the mechanism of interaction with the target.

Thioaptamers and other nucleic acid analogs (e.g. peptide nucleic acids (PNAs), methylphosphonates, etc.) are emerging as important agents in therapeutics, drug discovery and diagnostics. Three key attributes define the unique ability of (thio)aptamers to perform their essential functions: (1) they target specific proteins in physiological pathways; (2) their sequence and structure is not intuitively obvious from canonical biologics and oftentimes can only be deduced by combinatorial selection against their targets; and (3) they bind their targets with higher affinities than do naturally occurring nucleic acid substrates. Importantly, the backbone modifications of thioaptamers and their nucleic acid backbone analogs enable aptamers to be introduced directly into living systems with in vivo lifetimes many times greater than unmodified nucleic acids, due to their inherent nuclease resistance of the modified aptamers. The inherent nuclease resistance is extraordinarily important for their efficacy in use.

The term “gene silencing” as defined herein is used to describe the phenomenon of reduced or repressed translation of mRNA into a protein. Examples of thioaptamer mediated “gene silencing” include short ssDNA, ssRNA or dsRNA, that may vary from 15 to 70 nt long (for precursors) that repress protein expression by specific or non-specific degradation of mRNA and/or binding to the mRNA in a location, time and manner that inhibits the cellular translational complex from translating the mRNA into protein. Degradation may occur, e.g., by non-specific antisense DNA/RNA duplex formation and resulting RNase H-type RNA degradation or sequence specific DICER/RISC mediated mRNA degradation. The term “RNA interference” (RNAi) is defined herein as gene silencing by cleavage of perfectly complementary mRNA, which in mammals is mediated by 21-23 nt small, interfering RNAs (siRNAs) which are double-stranded, and which are produced by Dicer cleavage of long ds RNA, with the resulting siRNA incorporated into an RNA-induced silencing complex (RISC). As used herein, the term gene silencing also applies to mRNA repression of translation, in which the mRNA complementarity is imperfect but the thioaptamers of the present invention are able to repress (lower or eliminate) gene translation.

Table 1 summarizes the types of gene silencing that may be achieved using the thioaptamers of the present invention. For example, gene silencing may be by cleavage of perfectly complementary mRNA mediated not only by siRNAs, but also by 21-22 nt, single-stranded mRNAs. The thioaptamer may be designed and selected, e.g., based on the target strandedness of the message or the thioaptemer and may be double- or single-stranded, which the skilled artisan will recognize as the distinguishing characteristics between a mRNA and a siRNA.

TABLE 1
Summary of Thioaptamer Gene Silencing Described Herein

| | | | | Comple- | |
|---|---|---|---|---|---|
| | | | | mentarity | |
| | | | | to target | Silencing |
| Trigger | Strand | Length | Precursor | Required | mechanism |
| siRNA | Ds | 21-23 nt | long | Perfect | mRNA |
| | | (mammals) | dsRNA | | cleavage |
| | | 21, 25 nt | | | |
| | | (plants) | | | |
| miRNA/ | Ss | 21-22 nt | 70 nt | Imperfect | repress |
| stRNA | | (eukaryotes) | stem-loop | | translation of |
| | | | RNA | | mRNA to |
| | | | (shRNA) | | protein |
| miRNA | Ss | 21-22 nt | 70 nt | Perfect | mRNA |
| | | | stem-loop | | cleavage |
| | | | RNA | | |
| | | | (shRNA) | | |

siRNA = small, interfering RNA
miRNA = micro, interfering RNA
stRNA = small, temporal RNA
shRNA = short, hairpin RNA

The thioaptamers of the present invention may operate by transcriptional silencing through which mRNA is not produced by the gene target and by post-transcriptional silencing. Two examples of post-transcriptional gene silencing, include: (1) an mRNA that is produced by transcription but is then cleaved/degraded by an siRNA or mRNA before it can express protein; and (2) an mRNA that is produced by transcription and is not cleaved/degraded, but its translation into protein is repressed/regulated by binding of a mRNA to, e.g., its 3′-UTR. Gene silencing by repression of the translation of mRNA targets to protein by the thioaptamers described herein may be mediated by single-stranded microRNAs (mRNAs) which are 21-22 nt long and are homologous but not perfectly complementary to the target mRNA, bind to the 3′-UTRs of the target mRNA, and are produced by Dicer cleavage of circa 70 nt long (“short”) “hairpin” RNA precursors. As such, the thioaptamers and the libraries of thioaptamers described herein (e.g., the library of libraries) may include thioaptamer shRNA precursors that are then “processed” into the mature “gene silencing” thioaptamer by Dicer. Such imperfectly complementary mRNAs are also called “small, temporal RNA (stRNA).” mRNA repression of translation has been identified in plants, worms, flies and mammals. Precursor “gene silencing” thioaptamers may be single- or double-stranded.

A “target gene” as defined herein may be, e.g., a gene derived from the cell, a transgene (e.g., a gene construct inserted at an ectopic site in the genome of the cell), or a gene from a pathogen that is capable of infecting an organism from which the target cell is derived. Depending on the particular target gene and the dose of thioaptamer delivered, this process may provide partial or complete loss of function for the target gene. In some cases, gene silencing of a target gene may be a reduction or loss of gene expression in at least 99% of targeted cells.

Generally, gene silencing may be shown by the inhibition of gene expression such that the level of protein and/or mRNA product from a target gene in a cell is absent or reduced about 5, 10, 20, 30, 50, 75 80, 90 or even about 100% (i.e., an observable decrease within the limits of detection of the assay selected to measure gene silencing). Specificity of the thioaptamer refers to the ability of the thioaptamer to inhibit the target gene without manifest effects on other genes of the cell. The consequences of inhibition may be confirmed by examination of phenotypic changes (i.e., outward properties of the cell or organism) or by genotypic or biochemical techniques such as RNA solution hybridization, nuclease protection, Northern hybridization, reverse transcription, gene expression monitoring with a microarray, antibody binding, enzyme linked immunosorbent assay (ELISA), Western blotting, radioimmunoassay (RIA), other immunoassays, and fluorescence activated cell analysis (FACS). For thioaptamer-mediated inhibition in a cell line or whole organism, gene expression may be assayed by use of a reporter or drug resistance gene whose protein product is easily assayed. Reporter genes may include, e.g., acetohydroxyacid synthase (AHAS), alkaline phosphatase (AP), beta galactosidase (LacZ), beta glucoronidase (GUS), chloramphenicol acetyltransferase (CAT), green fluorescent protein (GFP), horseradish peroxidase (HRP), luciferase (Luc), nopaline synthase (NOS), octopine synthase (OCS), and derivatives thereof. Furthermore, the detection of gene silencing may even be by using multiple selectable markers are available that confer resistance to ampicillin, bleomycin, chloramphenicol, gentamycin, hygromycin, kanamycin, lincomycin, methotrexate, phosphinothricin, puromycin, and tetracycline.

Depending on the assay used to measure gene silencing using the thioaptamers of the present invention, quantitation of the amount of gene expression allows one to determine a degree of inhibition which is greater than about 5%, 10%, 20%, 25%, 33%, 50%, 60%, 75%, 80%, 90%, 95% or about 99% as compared to a target cell that has not been not treated according to the methods of the present invention. The thioaptamers disclosed herein may permit the use of lower doses of injected material and longer times after administration of, e.g., dsRNA thioaptamers resulting in the inhibition of a smaller fraction of cells (e.g., at least about 10%, 20%, 50%, 75%, 90%, or about 95% of targeted cells). Quantitation of gene expression in a cell may show similar amounts of silencing that depends on the level of accumulation of target mRNA and/or translation of target protein. For example, the efficiency of inhibition may be determined by assessing the amount of gene product in the cell: mRNA may be detected with a hybridization probe having a nucleotide sequence outside the region used for the inhibitory double-stranded RNA, or translated polypeptide may be detected with an antibody raised against the polypeptide sequence of that region.

The thioaptamers disclosed herein may be delivered as a double-stranded RNA thioaptamer, as a single self-complementary RNA thioaptamer strand (single-stranded RNA tioaptamer with a tertiary structure, e.g., hair-pin loops) or two complementary RNA thioaptamer strands (or DNA.RNA duplexes). RNA thioaptamer duplex formation may be initiated either inside or outside the cell. The thioaptamer may be introduced in an amount which allows delivery of at least one copy per cell. Higher doses (e.g., at least 5, 10, 100, 500 or 1000 copies per cell) of double-stranded thioaptamers may yield more effective inhibition; lower doses may also be useful for specific applications. Inhibition is sequence-specific in that nucleotide sequences corresponding to the duplex region of the RNA are targeted for gene silencing.

Thioaptamers having a nucleotide sequence identical to a portion of the target gene will most often be used for gene silencing, however, nucleotide sequences may be varied by insertions, deletions, and single point mutations relative to the target gene sequence. Thus, sequence identity may be optimized by sequence comparison and alignment algorithms known in the art that calculate the percent difference between the nucleotide sequences by, for example, the Smith-Waterman algorithm as implemented in the BESTFIT software program using default parameters (e.g., University of Wisconsin Genetic Computing Group (GCG)), ClustalW, etc. The thioaptamers may have a sequence identity greater than 90% with a target sequence, or even 100% sequence identity, between the inhibitory RNA and the portion of the target gene. Alternatively, the duplex region of the RNA may be defined functionally as a nucleotide sequence that is capable of hybridizing at medium to high stringency, as will be known to those of skill in the art (See e.g., Maniatis, et al.) with a portion of the target gene transcript (e.g., 400 mM NaCl, 40 mM PIPES pH 6.4, 1 mM EDTA, 50° C. or 70° C. hybridization for 12-16 hours; followed by washing). The length of the identical nucleotide sequences may be at least about 25, 50, 100, 200, 300 or 400 bases for the precursors. As such, 100% sequence identity between the thioaptamer and the target gene is not required to practice the present invention, which allows for tolerate of sequence variations that might be expected due to genetic mutations, polymorphisms, or evolutionary convergence, drift, shift and divergence.

A cell with the target gene may be derived from or contained in any organism or particle. The organism may a plant, animal, protozoan, bacterium, virus, or fungus. The plant may be a monocot, dicot or gymnosperm; the animal may be a vertebrate or invertebrate. Microbes may be, e.g., those used in agriculture or by industry, and those that are pathogenic for plants or animals. Fungi include organisms in both the mold and yeast morphologies. Particles may include viruses and the like.

The cell having the target gene may be from the germ line or somatic, totipotent or pluripotent, dividing or non-dividing, parenchyma or epithelium, cloned, immortalized or transformed and the like. The cell may be a stem cell or a differentiated cell and may be derived from a wild-type, a genetic mutant, a genotypic variant, a transgenic, a knock-out, a knock-in and the like. Cell types that are differentiated include, e.g., adipocytes, fibroblasts, myocytes, cardiomyocytes, endothelium, neurons, glia, blood cells, megakaryocytes, macrophages, granulocytes, e.g., neutrophils, eosinophils and basophils, mast cells, lymphocytes, e.g., B-cells and T-cells, keratinocytes, chondrocytes, osteoblasts, osteoclasts, hepatocytes, and cells of the endocrine or exocrine glands.

The thioaptamer may be directly introduced into the cell (i.e., intracellularly); or introduced extracellularly into a cavity, interstitial space, into the circulation of an organism, introduced orally, or may be introduced by bathing an organism in a solution containing the thioaptamer. For example, the thioaptamer may be sprayed onto a plant or a plant may be genetically engineered to express the thioaptamer in an amount sufficient to kill some or all of a pathogen known to infect the plant. Physical methods of introducing the thioaptamer may include, e.g., injection directly into the cell or extracellular injection into the organism. Other methods for delivering the thioaptamer include, e.g., bombardment by particles covered by the thioaptamer, soaking the cell or organism in a solution of the thioaptamer or electroporation of cell membranes in the presence of the thioaptamer. Other methods known in the art for introducing nucleic acids to cells may be used, such as lipid-mediated carrier transport, chemical-mediated transport, such as calcium phosphate, and the like. Thus the thioaptamer may be introduced along with components that perform one or more of the following activities: enhance thioaptamer uptake by the cell, promote annealing of the duplex strands, stabilize the annealed strands, or other-wise increase inhibition of the target gene.

The thioaptamer may also be used for the treatment or prevention of disease. For example, dsRNA thioaptamers may be introduced into a cancerous cell or tumor and thereby inhibit expression of a gene required for maintenance of the carcinogenic/tumorigenic phenotype. To prevent a disease or other pathology, a target gene may be selected which is required for initiation or maintenance of the disease/pathology. Treatment would include amelioration of any symptom associated with the disease or clinical indication associated with the pathology.

The thioaptamers may also target a gene for immunosuppression of a host. Alternatively, the thioaptamers may be targeted at target genes for replication of a pathogen, transmission of the pathogen, or maintenance of the pathogenic infection. The gene silencing thioaptamer is introduced in cells in vitro or ex vivo and then subsequently placed into an animal to effect therapy, or directly introduced by in vivo administration.

Research studies comparing siRNA to chemically optimized antisense technology, have indicated that fewer RNA duplexes have to be screened in order to identify active siRNAs, that siRNAs might be more potent inhibitors of gene expression than antisense (Miyagishi, et al., 2003), and that siRNAs were less toxic to cells (Braasch, et al., 2003). Phosphorothioate (PS) modified antisense oligonucleotides are the “gold standard’ for antisense therapy, conferring nuclease resistance to these ss DNA oligonucleotides and increasing binding to serum proteins which increases bioavailability (Geary, et al., 2001). A next generation of phosphorothioate anti-sense oligonucleotides may be based on the introduction of locked nucleic acid bases (LNA) into the molecule to enhance binding affinity (Braasch and Corey, 2002).

Researchers in the field have studied the effects of similar chemical modification of siRNA for use in RNAi to potentially enhance serum and cellular stability. Partial substitution of dsRNA with either phosphorothioate linkages or 2′-deoxy-2′-fluorouridine nucleotides, on one or both strands has been shown to continue to support RNAi (Parrish, 2001). Whereas extensive 2′-O methyl modification did not support RNAi (Elbashir, 2001b), limited modification did support RNAi (Amarzguioui, 2003). The fluorouridine modification may have an added advantage in preparation of the large amounts of material required by a therapeutic application, in that elimination of the 2′-hydroxyl group simplifies synthesis, deprotection and purification protocols. It has also been observed that whereas minimal substitution with phosphorothioate linkages is tolerated, extensive phosphorothioate substitution is toxic to cells (Prydz, 2003). However, in a separate study with PS modification at 4-21 nucleotides of a 21 nt siRNA, cell toxicity was not observed (Braasch, et al., 2003). The discrepancy between the two reports was ascribed by Braasch as possibly being due to the presence of toxic impurities in the PS-siRNA samples used in the Prydz work. Importantly, there is no method to predict the optimal location of the thiophosphate modification to optimize siRNA activity and serum and cellular stability.

Research on the effect of a single mutation in a siRNA strand on RNAi activity has indicated that while a mutation on the antisense strand reduces activity, a mutation on the sense strand has no effect. This indicates that the two strands of a siRNA molecule have different functions in RNA interference. Chemical modification of the 3′ end of the sense strand exclusively is possible without incurring loss of RNAi activity. However chemical modification at the 3′ end of the antisense strand abolished activity, indicating that it is the 3′ end of the antisense strand that is recognized by the RISC (Hamada, et al., 2003). The following is a summary of the research on the stability and efficacy of PS-siRNA in mammalian cell culture (Braasch, et al., 2003).

Stability. Surprisingly, whereas ssRNA is degraded rapidly by serum nucleases, dsRNA was reasonably stable in serum. Since siRNA must remain intact in order to interact with the RISC complex, the dsRNA nuclease resistance presumably facilitates endogenous RNAi. PS-ssRNA substituted with 12 or 21 (complete) PS linkages was degraded in serum. Finally, PS-modified dsRNA was stable in serum, although its stability was not significantly higher than that of unmodified dsRNA.

Efficacy. Modification with either PS, fluorouridine or LNAs continues to support RNAi and introduction of PS linkages into dsRNA reduced Tm (78° C. to 58-73° C.), whereas fluorouridine substitution did not affect Tₘ and LNA substitution increased Tm significantly (78° C. to 93° C.). Braasch hypothesized that siRNA thermal stability might be important since lower Tm could cause dissociation and since ssRNA is degraded by nucleases, could reduce RNAi. The Tm for all cases of substitution, however, is significantly higher than physiological temperature and such hypothesized dissociation may be negligible. Reduction of the melting temperature of the dsRNA, while still keeping Tₘ well above physiological temperature, could enhance siRNA activity if unwinding of the duplex is a limiting step in the enzymatic reaction sequence of RNAi. Since dithiophosphate linkages reduce the melting temperature of the siRNA/thioaptamer more than monothiophosphates, the dithiophosphates could be optimal for chemical modification of siRNA. Furthermore, although greater than 80% of cells were successfully transfected with both unmodified and PS-modified dsRNA, the nuclear uptake of PS-modified dsRNA was significantly higher than that of unmodified dsRNA, consistent with an earlier report that PS-DNA/lipid complexes exhibited enhanced nuclear localization (Marcusson, 1998). Finally, PS-dsRNA with 4, 8, 12, 16 and 21 PS linkages inhibited gene expression at low levels (sub-50 nM) whereas unmodified dsRNA activity dropped significantly below 50 nM.

Although in the study described above, PS-dsRNA did not significantly increase serum stability of the already stable dsRNA, it was hypothesized by the authors that the PS linkages may improve the pharmokinetics of siRNA (Braasch, et al., 2003), since modification with as few as 13 PS substitutions improves the pharmacokinetics of antisense oligonucleotides by increasing their binding to serum proteins (Geary 2001). It was thus proposed that a few LNA modifications, avoiding the central region of the siRNA, in order to increase thermal stability, should be combined with PS-linkages to improve pharmokinetics as a strategy for design of a chemically modified siRNA.

Whereas the aforementioned study indicated that PS-siRNA did not enhance nuclease resistance, at least one developer of siRNAs has argued that without chemical modifications RNAi will not be an option for therapeutics. The same developer has claimed that the ribose sugar makes the siRNA molecule unstable, and thus they have replaced it with a “ribose prosthetic,” e.g., xylofuranosyl modifications into polynucleotides (Matulic-Adamic, et al., 1996). In vitro studies of hepatitis B virus (HBV), by Sirna Therapeutics, e.g., found that standard siRNA was more active than “prosthetic” siRNA at day three post-administration, but the “prosthetic” siRNA was much more active at day 21 post-administration (comments in RNAi Roundup article on GenomeWeb internet site). Sequitur Inc., developer of a “Stealth RNAi,” has found that standard siRNA molecules transfected into cells undergo degradation in the cytosol within hours, although the triggered RNAi activity persists for days. The so-called “Stealth” RNAi is said to be significantly more stable in the cytosol, allowing the monitoring of siRNA uptake. The inventors of the subject invention believe that these studies indicate a need for increasing the nuclease resistance and in vivo stability of siRNA, as this could enhance dose persistence in a therapeutic setting.

While others have used these so-called “prosthetic” modifications, the present inventors have reasoned that the enhanced nuclear localization of PS-dsRNA should also increase therapeutic efficacy and the higher RNAi activity at dsRNA levels below 50 nM may also be significant in vivo. Using the present invention, a variation in the number of LNA substitutions and the location of LNA substitutions on a 21 nt siRNA resulted in significant variation in inhibition of gene expression, hypothesized to be due to variation in the ability of the modified LNA-siRNA to be recognized by the proteins comprising the RISC complex (Braasch, et al., 2003). Variation in the number of PS linkages and their position on a 21 nt siRNA targeting human Tissue Factor resulted in significant variation in persistence of gene silencing after 5 days post-transfection with siRNA, whereas PS-siRNA and wildtype-siRNA exhibited similar activity before day 5 (Amarzguioui, 2003).

Extensive work on thioaptamer recognition of proteins by the inventors of the subject invention, suggests that combinatorial library selection of PS-siRNA, in which the nucleotide sequence is constant, but the number and position of the PS links are combinatorially varied, or in which both backbone and sequence are varied, should yield optimized siRNA for specific applications, and thus for specific RISC complex recognition. Combinatorial library selection allows the identification of thiophosphate patterns that enhance the cellular nuclease stability of the thioaptamer, enhance binding to RISC proteins and catalytic activity in the RISC-siRNA nuclease cleavage of the mRNA transcript. Recognition of the siRNA molecule by the proteins included in the RISC complex is required in order that the separation of the siRNA strands and subsequent recognition of the mRNA target by an siRNA strand can proceed. Current means of optimization of siRNA depend on algorithms based on sets of selection rules, focusing on siRNA sequence in terms of optimum sites on the target gene and avoidance of sites common to a family of proteins and to sites known to activate interferon response—examples are Dharmacon's 34 rule algorithm (www.dharmacon./com) and/or Tushl's set of selection rules.

The present invention uses a novel methodology of combinatorial library selection of aptamers in order to select small (30 nt random sequence region), partially thioated RNA aptamers targeting the Venezuelan Equine Encephalitis (VEE) virus. VEE is a likely agent of biological warfare and/or terrorism (BWT) for several reasons:
(1) VEE virus is known to have been highly developed as a BWT agent in both the United States and in the former Soviet Union.
(2) VEE virus is readily isolated from natural sources.
(3) VEE virus replicates to high titer in a variety of cell cultures.
(4) VEE virus is highly stable when lyophilized.
(5) VEE virus is highly infectious by aerosol; over 150 instances of laboratory aerosol infection have been documented.
(6) VEE virus produces a highly debilitating and sometimes fatal disease, with permanent neurological sequelae in many cases.
(7) If introduced into a location with susceptible equines and mosquitoes, VEE virus can produce a widespread epidemic.
(8) No licensed VEE virus vaccine exists. Current experimental vacines have poor efficacy and a high rate of adverse reactions.
(9) No effective antivirals have been developed against VEE virus.

Because VEE virus may be used for biological terrorism, the present invention includes new strategies for antiviral development, focusing on powerful combinatorial methods that allow for rapid selection and identification of lead compounds for cell culture and animal challenge studies. The present invention includes compositions and methods for the rapid isolation, identification, purification, characterization and development of gene silencing thioaptamers, thiophosphate-backbone modified oligonucleotide agents (RNA- and DNA-based oligonucleotides (ODNs)) to a wide range of proteins and mRNA, including viral proteins that are essential for virion assembly and/or the mRNA transcripts which translate to viral proteins.

The VEE virus capsid protein is an attractive target protein because it interacts with other capsid protein molecules in nucleocapsid formation, and also interacts with the cytoplasmic tail of the E2 envelope glycoprotein to initiate virion budding. Thus, the initial studies focused on targeting the capsid protein using the new combinatorial selection technology. The in vitro combinatorial selection methodology has selected ssRNA thioaptamers towards the nucleocapsid protein of VEE virus (all monothiophosphates at the 5′-dA positions). Combinatorial selection of RNA aptamers targeting VEE virus was implemented with completion being assessed based on convergence of sequence of RNA aptamers with an affinity of 1-2 nM. Table 2 shows the aligned sequences of 23 high affinity thioRNA aptamers (random sequence region is 30 nt), which share considerable sequence identity.

TABLE 2
is a ClustalW Multiple Sequence Alignment of RNA Aptamers

| | | |
|---|---|---|
| 7-2 | -CUGCCUACG-CCAUGCCCAGAACCCUCACGC-- | (SEQ ID NO.: 1) |
| 13-A10 | -CUGCCUACG-CCAUGCCCAGAACGCUCACGC-- | (SEQ ID NO.: 1) |
| 16-2 | -CUGCCUACG-CCAUGCCCAGAACCCUCACGC-- | (SEQ ID NO.: 1) |
| 16-4 | -CUGCCUACG-CCAUGCCCAGAACCCUCACGC-- | (SEQ ID NO.: 1) |
| 16-8 | -CUGCCUACG-CCAUGCCCAGAACCCUCACGC-- | (SEQ ID NO.: 1) |
| 16-16 | -CUGCCUACG-CCAUGCCCAGAACCCUCACGC-- | (SEQ ID NO.: 1) |
| 16-7 | -CUGCCUACG-CCAUGCCCAGAACCCUCACGC-- | (SEQ ID NO.: 1) |
| 16-5 | -CUCCCUACG-CCAUGCCCAGAACCCUCACGC-- | (SEQ ID NO.: 1) |
| 7-7 | GGCCCUUGCGCCCACACGCAAACACCGCCC---- | (SEQ ID NO.: 2) |
| 7-11 | GGCCCUUGCGCCCACACGCAAACACCGCCC---- | (SEQ ID NO.: 2) |
| 16-1 | GGCCCUUGCGCCCACACGCAAACACCGCCC---- | (SEQ ID NO.: 2) |
| 7-12 | -CGCCAACCGACCGUCCCGACCGUCCGCCUC--- | (SEQ ID NO.: 3) |
| 16-9 | -CGCCAACCGACCGUCCCGACUGUCCGCCUC--- | (SEQ ID NO.: 3) |
| 16-12 | --UGCCCAGG-CCGCGGCCAUCACUACUACGCC- | (SEQ ID NO.: 4) |
| 13-A9 | GCUCAGAUCCCCCCGCCCCGCUAUCCGCAC---- | (SEQ ID NO.: 5) |
| 13-A11 | ---UCCUGUGCCCGGACCCUGUCCCCUGGUG--- | (SEQ ID NO.: 6) |
| 16-6 | --UGCCAA-GUCGGCUUCCAUCCACCACCCGAG- | (SEQ ID NO.: 7) |
| 16-15 | ----CCGACGGAAUUCCCGCUAGUUCCCCUGACC | (SEQ ID NO.: 8) |
| 7-14 | ----CCGACGAC--UGAUUAUUCCCCUGCCCCCA | (SEQ ID NO.: 9) |
| 16-14 | ---UCACACCACACGCUUCAUCCCCUGCAC---- | (SEQ ID NO.: 10) |
| 7-6 | -CACCUCACAACUUCGCACCUCAACCGUCUC--- | (SEQ ID NO.: 11) |
| 7-9 | ----CCCUCAGCACUUGCUUGCCAGCGGACGCCA | (SEQ ID NO.: 12) |
| 7-10 | AUGGCUUACAAGCCGCAGCUGUAUGUGGAG---- | (SEQ ID NO.: 13) |

Although statistically significant homology cannot be established between any of the selected aptamer sequences and the VEE virus genome, significance would not be expected unless the identity is circa 100% for these short sequences. More importantly, cis-acting RNA sequences important for VEE viral replication generally are defined by secondary structure rather than by primary sequences, so homology would not necessarily be expected.

Initial testing of aptamer No. 7-7 did not reveal any evidence of antiviral activity (data not shown), however, a positive control for liposome fusing and RNA entry was not available. Synthesis of a fluorescein-labeled aptamer for evaluating RNA uptake and localization in cells before doing more challenge studies may be used in conjunction with the aptamer to optimize delivery of the aptamer. Once delivery is optimized, the antiviral activity in Vero and BHK cells may be detected, and the studies in a mouse model of antiviral activity finalized.

### EXAMPLE 1

Combinatorial selection and characterization of phosphorothioate RNA aptamers against VEE capsid protein. Combinatorial selection of aptamers was employed to isolate RNA aptamers targeting VEE (Venezuelan Equine Encephalitis) virus capsid protein. VEE is a potential bioterrorism agent, and its capsid protein, which plays a major role in viral replication, is a drug target. The combinatorial selection procedure was designed to modify the backbone of RNA aptamers with phosphorothioate linkages. This chemically modified phosphorothioate RNA (PSRNA or thioaptamer) is expected to improve the efficiency and stability of a RNA aptamer as a potential drug. One of the highest affinity thioRNA aptamers from the first generation selection was aptamer 16_{—}1:

(SEQ ID NO.: 14)

5′-GGGAGCUCAGAAUAAACGCUCAAGGCCCUUGCGCCCACACGCAAAGA
CCGCCCUUCGACAUGAGGCCCGGAUCCGGC-3′ (30 nt random
region is underlined).

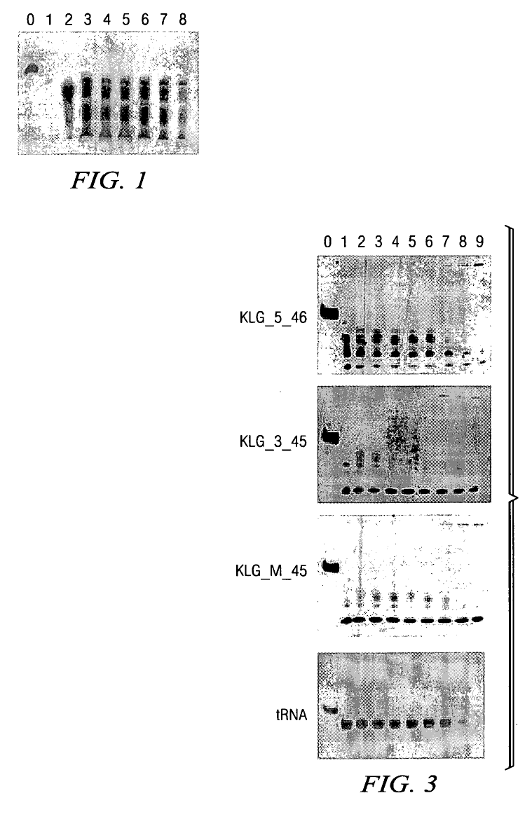

To develop a second generation aptamer, it was necessary to perform structural mapping (i.e., footprinting) to elucidate the VEE capsid binding site on the aptamer. FIG. 1 is a footprinting gel that shows the binding of 16_{—}1 to the VEE Capsid protein. The chemical footprinting of aptamer 16_{—}1 was conducted as follows: 10 nM of biotinylated aptamer 16_{—}1 was incubated with variable concentration of VEE capsid protein: Protein concentration of each lane was 0 (lane 4), 1 nM (lane 5), 19 nM (lane 6), 100 nM (lane 7), 1 mM (lane 8) and 10 mM (lane 9). After 2 hours incubation, iodine and ethanol mixture was added to the binding mixture to cleave unprotected phosphorothiolated phosphate bonds in the RNA aptamer. Lane 1 is a protein size marker. Lane 2 is aptamer 16_{—}1 only. Lane 3 is aptamer 16_{—}13.

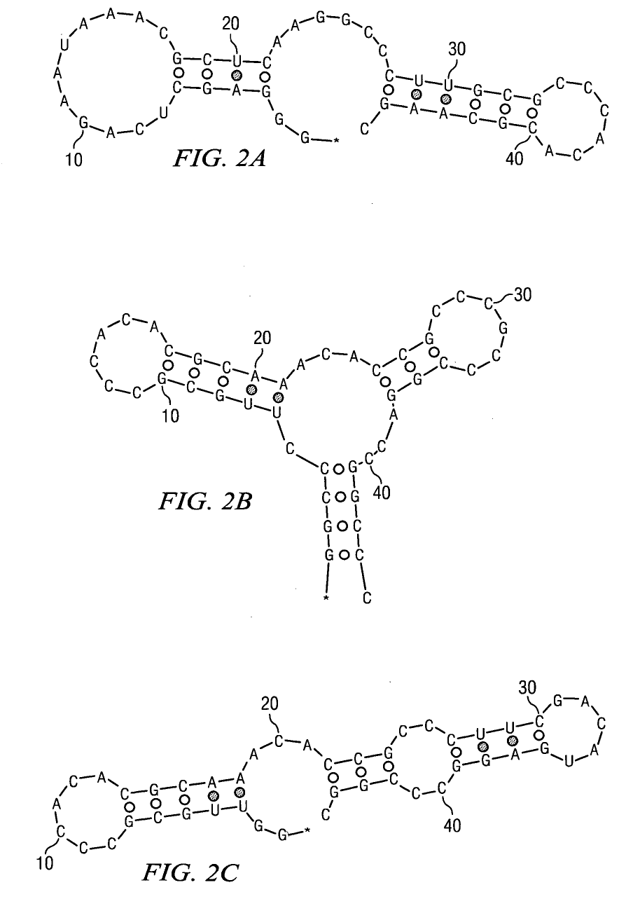

According to the footprinting result there is no isolated region on the aptamer that binds to VEE capsid protein. To determine the structural region on the thioRNA aptamer that is essential and sufficient for binding, three engineered RNAs were made (FIGS. 2A, 2B, 2C) from the aptamer 16_{—}1 and tested their binding capability. Each RNA migrated to show multiple bands in the gel.

KLG_5_46:

(SEQ ID NO.: 15)

5′-GGGAGCUCAGAAUAAACGCUCAAGGCCCUUGCGCCCACACGCA
AGC-3′
KLG 3_45:

(SEQ ID NO.: 16)

5′-GGCCGUUGCGCCGACACGCAAACACCGCCCGCCCGGAUCC
GGCC-3′
KLG_M_45:

(SEQ ID NO.: 17)

5′-GGUUGCGCCCACACGCAAACACCGCCCUUCGACAUGAGGC
CCGGC-3′

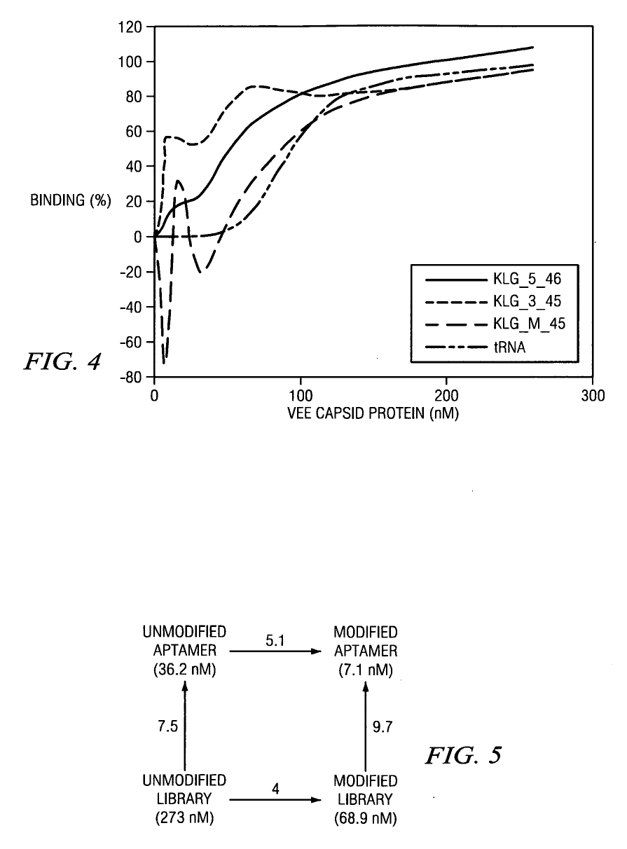

As shown in the gels in FIG. 4, only the upper bands of each RNA bound to VEE capsid protein. The binding assay of three engineered RNAs was as follows: 0.5 nM of biotinylated RNA was incubated with variable concentrations of VEE capsid protein. Protein concentration of each lane was: 0 nM (lane 3), 4 nM (lane 4), 8 nM (lane 5), 16 nM (lane 6), 32 nM (lane 7), 64 nM (lane 8), 128 nM (lane 9) and 256 nM (lane 10). After 2.5 hours incubation, the binding mixture was loaded onto the gel. Lane 1 is a protein size marker. Lane 2 is the same as Lane 3 but with 25% formamide added to partially denature the RNA. Binding of the RNA was measured based on the decrease of the upper bands as protein concentration was incremented. From this analysis, KLG_{—}3_{—}45 was determined to be the tightest binding aptamer. Based on this result, second generation RNA aptamers may be selected based on modifications of KLG_{—}3_{—}45.

The first generation selection procedure was studied from a system point of view, to characterize the degree of selection achieved by the combinatorial selection procedure. Comparison of the apparent binding constants of phosphate and phosphorothioate forms of the initial library and of the combinatorially selected aptamer 16_{—}1 to the VEE capsid protein indicated that: (1) selection did not significantly enhance the affinity of unmodified RNA to VEE capsid protein. This can be explained from the fact that VEE capsid protein binds nucleic acids promiscuously; (2) the position of the phosphorothioate modification is a key determinant in selection. Different enhancement of affinity to VEE capsid protein due to the phosphorothioate modification between the initial library and selected aptamer indicate the position of phosphorothioate in the selected aptamer played a role in the selection of the aptamer. For example, the PS-aptamer selected from the PS-library had 5.1-fold higher affinity than the unmodified aptamer selected from the unmodified library, and the PS-aptamer selected had 9.7 times higher affinity than the library whereas the unmodified aptamer had 7.5 times the affinity of its library. These results allow the generation of a model for selection and modification (FIG. 5).

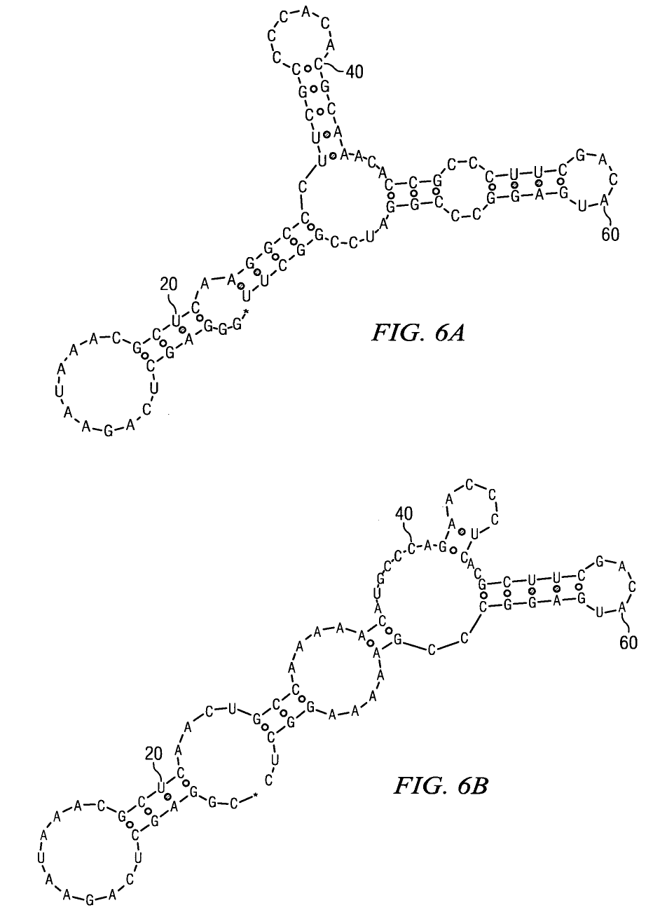

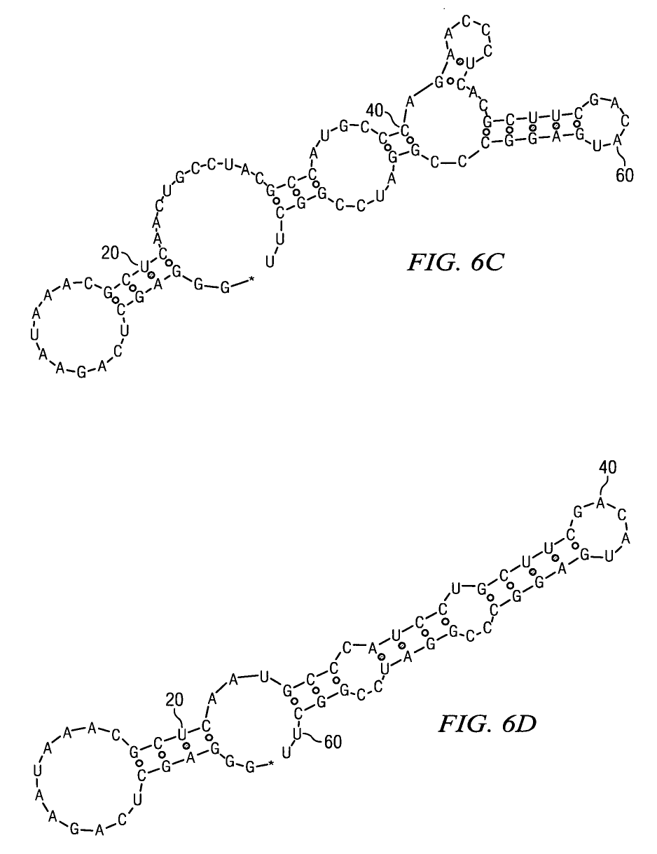

Three further variants of aptamer 16_{—}1 were developed and the stem-loop structures determined shown in FIGS. 6A, 6B, 6C and 6D:

FIG. 6A:

(SEQ ID NO.: 18)

5′-GGGAGCUCAGAAUAAACGCUCAAGGCCCUUGCGCCCACACGCAAACA
CCGCCCUUCGACAUGAGGCCCGGAUCCGGCUU-3′
FIG. 6B:

(SEQ ID NO.: 19)

5′-GGGAGCUCAGAAUAAACGCUCAACUGCCUACGCCAUGCCCAGAACCC
UCACGGUUCGACAUGAGGCCCGGAUCCGGCUG-3′
FIGS. 6C:

(SEQ ID NO.: 20)

5′-GGGAGCUCAGAAUAAACGCUCAACUGCCUACGCCAUGCCCAGAACCC
UCACGCUUCGACAUGAGGCCCGGAUCCGGCUU-3′
FIG. 6D:

(SEQ ID NO.: 21)

5′-GGGAGCUCAGAAUAAACGCUCAAUGCCGAUCCUGC
UUCGACAUGAGGCCCGGAUCCGGCUU-3′

The underlined portions show the variance from the 16_{—}1 aptamer, which also contain two new residues at the 3′-end.

Additional thioRNA aptamers targeting the VEE virus capsid protein will be tested for antiviral activity in cell cultures and in animal models. Additional VEE virus targets may also be studied, using the combinatorial selection/thioation methodology. These studies are described below and are illustrative.

The E2 envelope glycoprotein as aptamer target. This protein resides on the tip of the virion spikes, while E1 lies parallel to the envelope (Lescar et al., 2001; Pletnev et al., 2001). E2 is the site of the major antigenic determinants including most neutralizing epitopes, and is likely to interact with cellular receptors like the high affinity laminin receptor (Griffin, 2001). Therefore, E2 represents the best target for disruption of virion binding and entry into cells. To target E2 with thioRNA aptamers, it is possible to: (1) express the extracellular portion of the E2 protein using E. coli in a maltose binding protein fusion form, purified with an amylose column, or using the baculovirus system to preserve glycosylation; and/or (2) isolate E2 from purified VEE virus virions using weak (non-denaturing) detergent treatment followed by affinity column purification or isoelectric focusing column purification. If necessary, digestive removal of the transmembrane and cytoplasmic portions may be used.

The aptamer selection strategy will be essentially the same that the inventors have used for targeting the VEE virus capsid protein. This in vitro combinatorial selection technology is described in detail in a study of selection of aptamers targeting proteins such as NF-kB (King et al., 2002) and in co-pending application Ser. Nos. 07/430,733; 09/425,798; 09/425,804; 10/120,815; 10/214,417 and 10/272,509, relevant portions, sequences and/or thio-modification(s) incorporated herein by reference.

Cis acting RNA sequences in the VEE virus genome. The three cis-acting sequences that are highly conserved among alphaviruses and are believed to interact with VEE virus nonstructural proteins and cellular proteins for viral replication may be targeted by the thioaptamers of the present invention (Schlesinger and Schlesinger, 2001; Strauss and Strauss, 1994). For example, a combinatorial library of RNA thioaptamers may be produced that target those highly conserved regions of this or any other virus for identification and selection.

The 5′ end of the VEE virus genome contains two highly stable stem loop structures that are conserved in their secondary structure. These may also be targeted using the thioaptamers of the present invention. Mutagenesis studies to ablate the stem-loops yet preserve the amino acid sequence in the nsP1 protein render the virus noninfectious, confirming the importance of these secondary structures (I. Frolov, S. Weaver, unpublished).

The 26S subgenomic promoter. This sequence is also strongly conserved among togaviruses (including rubella) and presumably interacts with the polymerase for initiation of transcription. The 3′ untranslated genome region is highly conserved among alphaviruses and interacts with cellular proteins including the La antigen. These three RNA elements will be used to make direct decoys with high affinity and stability conferred by limited thiolation. The inventors will also introduce both monothiophosphate and dithiophosphate backbone substitutions in various random positions in the loop region of the RNA elements to enhance the aptamer affinities. These thioaptainers will be tested in a high throughput screening of inhibition of virus replication.

Combinatorial libraries of aptamers may also be attached to small polystyrene beads (one aptamer sequence per bead) to select for binding to whole VEE virus virions. Purified TC-83 virus will be mixed with bead libraries and, following washing, flow cytometry in combination with anti-E2 monoclonal antibodies will be used to sort beads with high affinity virus binding. The selected pool of beads will be used to amplify a new, enriched library for subsequent rounds of selection. Finally, the selected aptamers will be tested for in vitro and in vivo antiviral activity.

Testing of aptamers for antiviral activity. Initial testing may be done in cell culture using TC-83 attenuated VE virus. For aptamers targeted against the E2 protein or whole virus, a mix of a range of aptamer concentrations may be used along with varying amounts of a virus inoculum may be used and compared with controls, e.g., a scrambled sequence, negative control aptamers, and infect Vero cells with virus at a Multiplicity of Infection (MOI) of 0.1. Following triplicate infections, one step growth curves may be compared with negative controls for detecting significant suppression of virus replication. Antiviral activity may be confirmed with repeat assays and cell specificity can be determined using other cells such as BHK, 293, HeLa, etc. For the thioaptamers designed to directly mimic conserved cis-acting sequences, delivery may be via lipofectin or other cationic liposomes for introduction into the cell cytoplasm. Approximately 5×10⁴ cells per well (24 well plates) may be seeded one day before the transfection studies. TfXTM-10 (liposome) transfection reagent (Promega) containing the cationic lipid component may be used according to the manufacturer's protocol. Ratios of 2:1 and 4:1 of liposome to nucleic acid were used for delivery of thioaptamers and this should be appropriate for delivery of RNA. A 400 μl volume of nuclease-free water may be added to the vial of liposome reagent and vortexed for 10 seconds to suspend the lipid film. The vial may then be heated to 65° C. in a water bath for 1 minute. After vortexing again, the vial may be stored at −20 C° overnight. TC-83 containing 1×104 PFU/100 μl will could be added to each well for 1 hour at 37° C., washed 2× with PBS, and then RNA aptamer or thioaptamer (a range of RNA concentrations will to be tested) may then be added to each well (3 replicates for each RNA thioaptamer or RNA aptamer concentration). The plates may be returned to the incubator for 1 hour and 1.25 ml of warm, complete medium may be added to each well. The culture medium may be sampled at 0, 8, 24 and 48 hours and the virus titer will be determined by standard plaque assay. For any aptamers that exhibit antiviral activity in vitro, a mouse model may be used to test for protection against lethal challenge with the virulent Trinidad donkey strain (parent of TC-83). The methods for in vivo delivery are being optimized in the arenavirus project.

An example of the application of the combinatorial library and selection methods to an assay allowing selection of a thio-siRNA is as follows: a split synthesis combinatorial chemistry method is to be developed to create a combinatorial library of [S₂]-ODN agents or mixed [O]/[S]/S₂]-backbone ODN. A library can also be made in which the backbone is varied combinatorially but not the sequence and/or a library in which both backbone and sequence is varied combinatorially. Each unique member of the combinatorial library is to be attached to a separate support bead.

### EXAMPLE 2

Synthesis of a one bead-one monothioRNA library. Standard phosphoramidite (DNA and RNA) chemistry was used for the monothio-RNA library. A 0.5M ¹H-tetrazole in acetonitrile was used as DNA activator. A 0.5M solution of DCI (dicyanoimidazole) in acetonitrile was used as RNA activator. The libraries were prepared on a 1 umole scale of polystyrene beads (66-70 um). The 15-mer downstream and upstream primers, 5′-d(GGATCCGGTGGTCTG)-3′ (SEQ ID NO.: 22) and 5′-d(CCTACTCGCGAATTC)-3′ (SEQ ID NO.: 23) were synthesized in parallel on a two-column DNA synthesizer (Applied Biosystems Expedite 8909). Following the 5′-primer, the 31-mer sequences programmed on the synthesizer for the combinatorial monothio-RNA library.

The following RNA thioaptamers were synthesized and used for the following studies: 5′-r(GA*UC*CU*GA*AA*CU*GU*UU*UA*AG*GU*UG*GC*CG*AU*C)-3′ (SEQ ID NO.: 24) on column 1 and 5′-r(cU*aG*gA*cU*uG*gC*aC*aA*cC*gU*cA*cA*cU*gC*uA*u)-3′ (SEQ ID NO.: 25) on column 2 (where a lower case letter indicates a 3′-thioate linkage, an upper case letter indicates a 3′-phosphate linkage and an asterisk indicates a position at which a “split and pool” occurred in order to synthesize the combinatorial region of the monothio-RNA).

The coupling yield was typically upwards of 98.5% as determined by the dimethoxytrityl cation assay. Sulfurization chemistry used the Beaucage reagent. The fully protected monothio RNA combinatorial library with the non-cleavable linker beads were treated with 4 ml of a mixture of 3:1 (v/v) (28%) NH3:EtOH at 39° C. for 21 hours. The beads were centrifuged, the supernatant was removed and the solid support was washed with double-distilled water. After lyophilization the solid support was treated with 2 ml of triethylamine hydrochloride (TEA-3HF) for 20 hours at room temperature. Again, the beads were centrifuged, the supernatant was removed and the solid support was washed with double-distilled water.

Several new approaches may be used to evaluate for assay/selection of thio-siRNAs. For example, consider a combinatorial library of thio-siRNAs targeting the GADPH gene, using an siRNA thioaptamer. Bead-bound combinatorial libraries (one bead-one thioaptamer) of the GADPH sequence siRNA are to be created using three alternate approaches:

(1) synthesize random monothioates in a split-pool chemically synthesized normal bead support and then put one or more beads into a well (of a 96 or higher multiple well-plate) and deprotect and release the deprotected RNA from the bead into the well. In some cases it may be useful to use a simple separation pack chromatographic purification of the ssRNA thioaptamer(s) in a well and then combining a group of several different combinatorial thioaptamers into another 96 well plate). One would then add just a normal phosphate complementary strand to each of the wells to convert to the ds siRNA thioaptamers. One would then add the liposome prep for cell delivery and then the rest of the Ambion kit for rapid testing of inhibition of GADPH protein translation (using Ambion immunochemistry kit). Control may be, e.g., Ambion-supplied siRNA without any thiophosphate in the RNA backbone. It is also useful to confirm that the iodoethanol cleavage of the thioaptamer siRNA works as demonstrated for DNA thioaptamers, followed by gel electrophoresis or mass spectrometry (MS/MS) fragmentation to identify where the thio locations on the thioaptamer are in each well.

(2) Alternatively, a ddRNA (DNA derived siRNA) derived may be used from DNA templates. A non-combinatorial bead may then be used with ATPαS an an NTP cocktail to transcribe the siRNA in one well, another NTP[αS] into another well. In one embodiment, varying ratios of ATP/ATP[αS] may be used to add thiophosphate and some normal phosphate at different A, G, T or U sites on the sequence.

(3) Use of a non-cell based assay for RNAi activity of a siRNA (Kawasaki, et al., 2003). A non-cell based siRNA assay may use either or both monothioate and dithioate siRNA combinatorial beads by directly synthesizing a bead based monothioate libraries, placing one bead in each well, adding the complementary strand, and then without need of liposome for cell delivery, just assay following the Taira et al (Kawasaki, et al., 2003) non-cell based method. A longer tether from the bead to the siRNA thioaptamer may be needed in some circumstances, e.g., PEG or a long UUUUUU tether.

It must first be determined whether the physical attachment of the ds-siRNA molecule on the bead prevents the Dicer endonuclease of the RISC complex from binding to the mRNA and ds-siRNA thioaptamer (a long linker might be required to minimize such interference). If interference was significant, one would then design a means of releasing the ds-siRNA thioaptamer from the one bead in the well. The user then monitors the cleavage of the target mRNA by mass spectrometry, gels or ribozyme type assays.

### EXAMPLE 3

Thioaptamer Gene Silencing. The following studies were conducted to demonstrate gene silencing using thioaptamers. Standard gene silencing studies were conducted with the following modification to the materials and methods. Phosphoromonothioate substituted siRNAs (thioaptamers) were used from the siSTARTER Luciferase Kit to conduct studies (Dharmacon, USA). The methods used were as described in the manufacturer's instructions. The term “luc” refers to the luciferase reporter gene. Briefly, the following siRNA duplexes were provided in the kit:

| | |
|---|---|
| Anti-luc siRNA-1 | |
| 5′-GAU UAU GUC CGG UUA UGU AUU | (SEQ ID NO.: 26) |
| UU CUA AUA CAG GCC AAU ACA U p-5′ | (SEQ ID NO.: 27) |
| Anti-luc siRNA-2 | |
| 5′-CUG AAU ACA AAU CAC AGA AUU | (SEQ ID NO.: 28) |
| UU GAC UUA UGU UUA GUG UCU U p-5′ | (SEQ ID NO.: 29) |
| Anti-luc siRNA-3 | |
| 5′-UCC GGA AGC GAC CAA CGC C UU | (SEQ ID NO.: 30) |
| UU AGG CCU UCG CUG GUU GCG G p-5′ | (SEQ ID NO.: 31) |
| Non-specific luc control siRNA | |
| 5′-AUG UAU UGG CCU GUA UUA G UU | (SEQ ID NO.: 32) |
| UU UAC AUA ACC GGA CAU AAU G p-5′ | (SEQ ID NO.: 33) |

The following siRNA thioaptamers duplexes were synthesized and used in these studies:

Thioluc1(thio siRNA duplex 1)

(SEQ ID NO.: 34)

5′- CU*G A*AU ACA AAU CA*C A*GA A UU -3′ (sense)

(SEQ ID NO.: 35)

3′-UU-GA C U UA UGU UUA GU G U CU UP-5′(antisense)
ThioLuc2 (Thio siRNA duplex 2)

(SEQ ID NO.: 36)

5′- C*UG *AAU ACA AAU C*AC *AGA A UU -3′ (sense)

(SEQ ID NO.: 35)

3′-UUGA C UUA UGU UUA GUG UCU UP-5′(antisense)
ThioLuc3 (Thio siRNA duplex 3)

(SEQ ID NO.: 37)

5′- *CU*G AAU ACA AAU CAC *AG*A A UU -3′(sense)

(SEQ ID NO.: 35)

3′-UU GA C UUA UGU UUA GUG UC U UP-5′(antisense)
ThioLuc4 (Thio siRNA duplex 4)

(SEQ ID NO.: 38)

5′- *CU*G AAU ACA AAU CA*C A*GA A UU -3′(sense)

(SEQ ID NO.: 35)

3′-UU GA C UUA UGU UUA GUG UC U UP-5′(antisense)
ThioLuc5 (Thio siRNA duplex 5)

(SEQ ID NO.: 39)

5′- C*UG *AAU ACA AAU CA*C A*GA A UU -3′(sense)

(SEQ ID NO.: 35)

3′-UU GA C UUA UGU UUA GUG UC U UP-5′(antisense)
ThioLuc6 (Thio siRNA duplex 6)

(SEQ ID NO.: 40)

5′- CUG *AA*U ACA AAU CA*C A*GA A UU -3′(sense)

(SEQ ID NO.: 35)

3′-UU GA C UUA UGU UUA GUG UC U UP-5′(antisense)
Control 1 (control thio siRNA duplex 1)

(SEQ ID NO.: 41)

5′- AU*G U*AU U GGCCU GU*A U*UA G UU -3′ (sense)

(SEQ ID NO.: 33)

3′-UUUA C A UA A CC GGA C AU A AU CP-5′
(antisense)

*= location of thio-modification.

Base sequences from thioluc 1 through thioluc 3 are the same as anti-luc siRNA-2 and the sequence of control 1 is the same as non-specific luc control siRNA. The plasmid used was pGL3 plasmid (pGL3-expression vector) in a 1× Universal buffer: 20 mM KCl, 6 mM HEPES-pH 7.5, 0.2 mM MgCl₂. Transfection was accomplished using a TransIT-TKO transfection reagent: 2.5 μg/μl of non-liposomal polymer/lipid formulation. All RNAs were dissolved in RNase-free water. For detecting luciferase activity, the Promega Steady-Glo Luciferase Assay Buffer and Promega Steady-Glo Luciferase Assay Substrate kits were used according to the manufacturer's instructions. HeLa cells were grown in Opti-MEM (GIBCO) and when needed, supplemented with DMEM with L-glutamine, pyridoxine hydrochloride, high glucose and without sodium pyruvate. Additionally, the following were added to the medium (500 ml/bottle) before using: 25 ml of 5% inactivated fetal bovine serum, 10.6 ml of 20 mM Hepes, 5 ml of 1.8 mM glutamine and 0.5 ml of 50.8 μM 2-mercaptoethanol. The HeLa Cells (human cervical epithelial adenocarcinoma, adherent; ccl-2 were obtained from ATCC, Rockville, Md. Other buffers used included 1×PBS buffer (GIBCO).

Cells were plated in 96-well plate in triplicate for each condition using 200 ul of 1×10^{u5} HeLa cells/ml suspension. The cells were incubated for 24 hours at 37° C. in 5% CO₂. siRNA or thioaptamer siRNA duplexes and plasmid were resuspended in as follows: 200 ul siRNA were resuspended in Universal Buffer to each siRNA tube for a final concentration of 1.0 uM. Next, 408 ul of Universal Buffer were added to each thioRNA tube for a concentration of 100 uM. For the thioaptamers, 2 ul of 100 uM thio siRNA duplex solution were added to 198 ul of the Universal Buffer to each tube for a final concentration of 1 uM. Finally, for the reporter plasmid, 40 ul RNase-free water were added to the 10 ug of reporter plasmid for a final concentration of 250 ng/ul and 40 ul RNase-free water was added to the 10 ug of reporter plasmid tube for a final concentration of 250 ng/ul. The above were vortexed briefly and centrifuged.

Formation of siRNA or thio siRNA-plasmid-lipid complex for transfection. Using all RNase-free solutions and tubes, the following mixtures were prepared in separate sterile polystyrene tubes:

TABLE 2A
TransIT-TKO/Plasmid dilution mixture (Mixture 1A)

| | | |
|---|---|---|
| Opti-MEM | 186.0 | ul |
| TransIT-TKO (2.5 ug/ul) | 10.0 | ul |
| Reporter Plasmid (250 ng/ul) | 4.0 | ul |

TABLE 2B
TransIT-TKO dilution mixture (Mixture 1B)

| | | |
|---|---|---|
| Opti-MEM | 37.2 | ul |
| TransIT-TKO (2.5 ug/ul) | 2.0 | ul |
| Rnase-free Water | 0.8 | ul |

Next, the following mixtures were created using Tables 3A-F as a guide, to create Mixtures 3A, 3B, 3C, and 3D (siRNAs or thioRNAs), Mixture 3E (plasmid alone), and Mixture 3F (background control) in sterile 1.5-2.0 ml tubes.

TABLE 3A
siRNA duplex 1 dilution mixture (Mixture 2A)

| | | |
|---|---|---|
| Opti-MEM | 29.7 | ul |
| siRNA duplex 1 (1 uM) | 3.3 | ul |
| Mixture 1A | 33.0 | ul |

TABLE 3B
siRNA duplex 2 dilution mixture (Mixture 2B)

| | | |
|---|---|---|
| Opti-MEM | 29.7 | ul |
| siRNA duplex 2 (1 uM) | 3.3 | ul |
| Mixture 1A | 33.0 | ul |

TABLE 3C
siRNA duplex 3 dilution mixture (Mixture 2C)

| | | |
|---|---|---|
| Opti-MEM | 29.7 | ul |
| siRNA duplex 3 (1 uM) | 3.3 | ul |
| Mixture 1A | 33.0 | ul |

TABLE 3D
Non-specific control duplex dilution mixture (Mixture 2D)

| | | |
|---|---|---|
| Opti-MEM | 29.7 | ul |
| Non-specific control (1 uM) | 3.3 | ul |
| Mixture 1A | 33.0 | ul |

TABLE 3E
Plasmid alone dilution mixture (Mixture 2E)

| | | |
|---|---|---|
| Opti-MEM | 29.7 | ul |
| Rnase-free water | 3.3 | ul |
| Mixture 1A | 33.0 | ul |

TABLE 3F
No Plasmid dilution mixture (Mixture 2F)

| | | |
|---|---|---|
| Opti-MEM | 29.7 | ul |
| Rnase-free water | 3.3 | ul |
| Mixture 1B | 33.0 | ul |

Each of these mixtures were mixed gently (not vortexed) and incubated at room temperature for 20 minutes. To these tubes, 264 ul of DMEM were added to mixture 2A, 2B, 2C, 2D, 2E and 2F tubes respectively and again mixed gently (not vortexed).

Transfections were carried out as follows, carefully remove the medium from the cells to be transfected, carefully wash the cells 2 times with 50 ul of 1×PBS and add 100 ul mixture 2A˜2F to each well of 96-well plate (in triplicate for each condition) respectively and gently rocked the plate back and forth for even distribution of reagent. The cells were then incubated with transfection reagent mixture for 48 hours at 37° C. in standard incubation conditions.

Luciferase Detection. Next, the entire contents of the Steady-Glo® Buffer were transfered to the bottle of Steady-Glo® Substrate at room temperature. The growth media from the cells was removed, being careful not to dislodge any of the cells. The cells were washed twice with 50 ul of 1×PBS buffer, again taking care not to dislodge any of the cells and 100 ul of the Steady-Glo® mixture was added to each well and incubates at RT for 5 minutes. Finally, the luminescence in a luminometer is measured. Each of the samples and controls were measured and the means of the triplicates were calculated as follows:
mean=(replicate 1+replicate 2+replicate 3)/3

The “No Plasmid” mean was subtracted from each sample (background subtraction) and divided by the background subtracted means by the “Plasmid Only” mean.

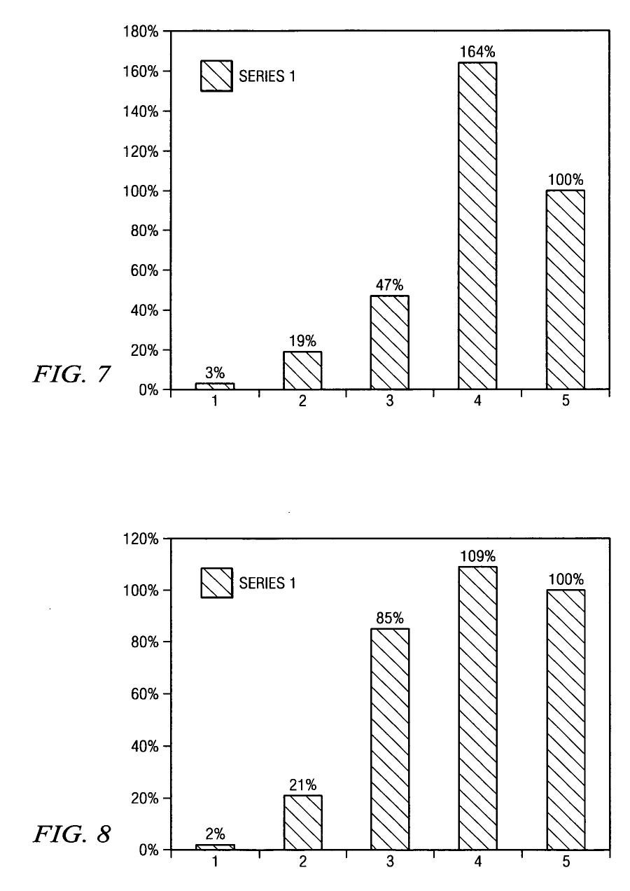

Phospho-siRNA silencing Studies. FIG. 7 is a graph of normalized mean luciferase activity in HeLa cells. Luminesence units were normalized to plasmid alone controls, and which the following were used:

| | | | | | |
|---|---|---|---|---|---|
| High | Medium | Low | Control | Plasmid | |
| silencer | silencer | silencer | siRNA | only | blank |

Raw Data:

| | | | | | | |
|---|---|---|---|---|---|---|
| Replicate | 1147 | 3647 | 8324 | 14564 | 12595 | 781 |
| 1 | | | | | | |
| Replicate | 1787 | 6519 | 11531 | 41383 | 33861 | 793 |
| 2 | | | | | | |
| Replicate | 1526 | 3519 | 10068 | 26587 | 14104 | 1247 |
| 3 | | | | | | |
| Mean | 1487 | 4561.7 | 9974.3 | 27512 | 20188 | 940.33 |

Blank subtracted

| | | | | | |
|---|---|---|---|---|---|
| Mean | 546.67 | 3621.37 | 9033.97 | 26571.67 | 19247.67 |

% silencing to plasmid alone

| | | | | | |
|---|---|---|---|---|---|
| Mean | 2.84 | 18.81 | 46.94 | 163.54 | 100 |

FIG. 8 is a graph that shows normalized mean luciferase activity in HeLa cells. Luminesence units were normalized to plasmid alone controls, and which the following were used:

| | | | | | |
|---|---|---|---|---|---|
| High | Medium | Low | Control | Plasmid | |
| silencer | silencer | silencer | siRNA | only | blank |

Raw Data:

| | | | | | | |
|---|---|---|---|---|---|---|
| Replicate | 1012 | 7231 | 13545 | 39057 | 1439 | 776 |
| 1 | | | | | | |
| Replicate | 2056 | 9817 | 38854 | 62856 | 66720 | 709 |
| 2 | | | | | | |
| Replicate | 839 | 4317 | 28603 | 897 | 26376 | 618 |
| 3 | | | | | | |
| Mean | 1302.3 | 7121.7 | 27001 | 34270 | 31512 | 701 |

Blank subtracted

| | | | | | |
|---|---|---|---|---|---|
| Mean | 601.3 | 6420.7 | 26300 | 33569 | 30811 |

% silencing to plasmid alone

| | | | | | |
|---|---|---|---|---|---|
| Mean | 1.95 | 20.84 | 85.36 | 108.95 | 100 |

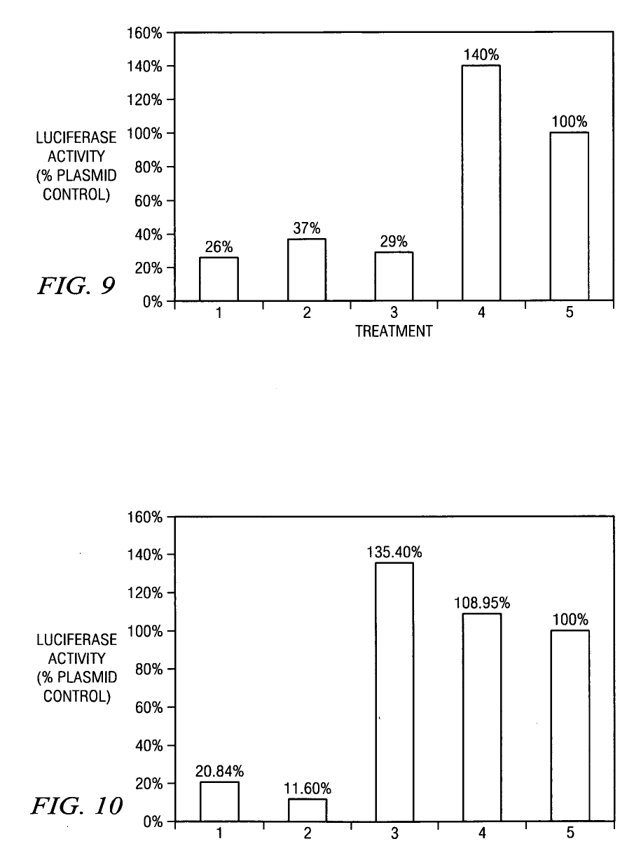

FIG. 9 is a graph that shows silencing by thioaptamers, background subtracted and normalized mean luciferase activity in HeLa cells. Luciferase activity units were normalized to plasmid alone control, and which the following were used:

| | | | control | | |
|---|---|---|---|---|---|
| thio | Thio- | Thio | thio | | |
| RNA | RNA | RNA | RNA | | |
| duplex | duplex | duplex | duplex | Plasmid | |
| 1 | 2 | 3 | 4 | only | blank |

Raw data:

| | | | | | | |
|---|---|---|---|---|---|---|
| Replicate | 1679 | 1777 | 1298 | 1718 | 1352 | 786 |
| 1 | | | | | | |
| Replicate | 1574 | 1733 | 2062 | 4624 | 3718 | 931 |
| 2 | | | | | | |
| Replicate | 1205 | 1640 | 1299 | 5115 | 3932 | 1143 |
| 3 | | | | | | |
| Mean | 1486.0 | 1716.7 | 1553.0 | 3819.0 | 3000.7 | 953.3 |

Blank subtracted

| | | | | | |
|---|---|---|---|---|---|
| Mean | 532.7 | 763.4 | 599.7 | 2865.7 | 2047.4 |

% silencing to plasmid alone

| | | | | | |
|---|---|---|---|---|---|
| Mean | 26.0 | 37.3 | 29.3 | 140.0 | 100 |

FIG. 10 is a graph that shows the effect of a thioaptamer on siRNA gene silencing in HeLa cells, and which the following were used:

1 phosphoro siRNA duplex 2

2 Thio-RNA oligo duplex 1

3 Thio-control RNA oligo duplex 4

4 Phosphoro control siRNA

5 plasmid alone

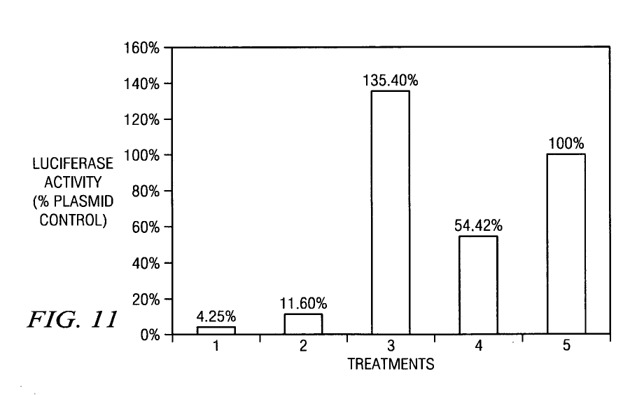

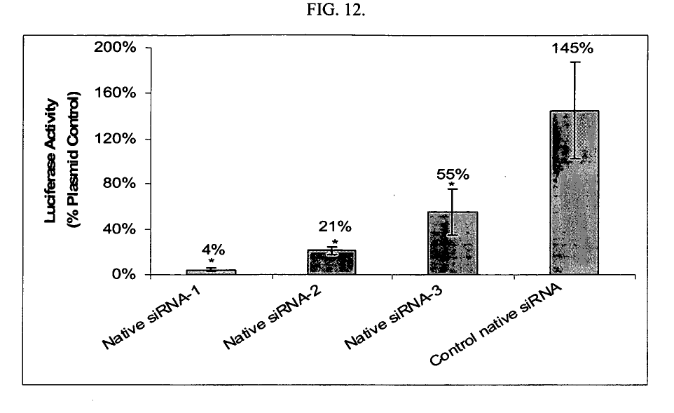

FIG. 11 is a graph that shows the effect of a thioaptamer on siRNA gene silencing in HeLa cells, and which the following were used:

| Phos- | | | Phos- | | |
|---|---|---|---|---|---|
| phoro- | Thio- | Thio- | phoro- | | |
| siRNA | RNA | control | control | | |
| duplex | duplex | RNA | siRNA | Plasmid | |
| 2 | 1 | duplex | duplex | only | blank |

Raw data:

| | | | | | | |
|---|---|---|---|---|---|---|
| Replicate | 965 | 912 | 4420 | 1545 | 4097 | 460 |
| 1 | | | | | | |
| Replicate | 1103 | 2407 | 18855 | 5028 | 13274 | 546 |
| 2 | | | | | | |
| Replicate | 918 | 1706 | 18147 | 11037 | 13637 | 610 |
| 3 | | | | | | |
| Mean | 995.33 | 1675 | 13807 | 5870 | 10336 | 538.67 |

Blank subtracted

| | | | | | |
|---|---|---|---|---|---|
| Mean | 416.66 | 1136.33 | 13268.33 | 5331.33 | 9797.33 |

% silencing to plasmid alone

| | | | | | |
|---|---|---|---|---|---|
| Mean | 4.25 | 11.60 | 135.4 | 54.42 | 100 |

One embodiment of the present invention includes thiophosphate substituted siRNA's that display gene silencing properties. Studies were conducted, in HeLa cell culture, thiophosphate siRNA and native siRNA Luciferase gene silencing studies, cytotoxicity studies and concentration-response studies using a Dharmacon siSTARTER Kit.

One embodiment includes a totally randomly thiophosphate substitutions that silenced gene activity, whereby demonstrating thioaptamer selection from a combinatorial library of partially thioated sequences using the Gorenstein et al bead-based approach is a process for selection of gene silencing aptamers.

Studies have shown a statistically significant difference in thiophosphate siRNA-1 luciferase gene silencing (5% activity) relative to that of an unmodified native siRNA-2 (17% activity) and to that of thiophosphate siRNA-2 (14% activity), indicating that (1) thiophosphate-modification of siRNA can enhance the silencing of luciferase gene expression compared with native siRNA of the same sequence, and that (2) thiophosphate siRNAs with the same nucleotide sequence but having different sulphur modification positions had different silencing effects.

One embodiment of the present invention incorporates thioations in different positions and in different whereby optimizing gene silencing activity. One embodiment of the present invention demonstrated a specific inhibitory effects of native siRNA-2 and thio siRNAs and demonstrated a concentration-dependent relationship over three or more orders of magnitude concentration, i.e., with concentration increase, the luciferase gene silencing effect also increased.

In the cytotoxicity studies, no significant changes were observed when HeLa cells were treated with 10 nM thiophosphate siRNAs. Lack of cytotoxicity is a benefit in terms of development of thio-siRNA aptamers for therapeutic purposes.

One embodiment of the present invention includes partial thio-modification of the siDNA analogs and of the RNA:DNA hybrid analogs of siRNA molecules to not only silencing genes with great specificity, but introduces the analogs into cells without transfection, whereby resulting in effectiveness against bacterial genes.

Preparation of siRNAs specific to luciferase gene. Native siRNA duplexes (phosphodiester backbone) were provided in Dharmacon siSTARTER (Product # K-002500-LU-01) and thio siRNA duplexes were designed by Gorenstein, et al. and synthesized by Dharmacon: On the basis of native siRNA-2, thio siRNAs were developed by placing sulphur modifications in different positions which were totally randomly selected from the sense strand of the native siRNA duplex-2, and using the same method, a control thio siRNA was developed on the basis of control native siRNA. The names and sequences of native siRNAs and thio siRNAs as well as the nucleotides with sulphur modifications in the thio siRNAs are shown in Table 4.

TABLE 4
Names and sequences of siRNA duplexes

| | | |
|---|---|---|
| Name | Sequences of nucleotides | SEQ ID NO.: |

| | | |
|---|---|---|
| Native siRNA duplexes | | |
| Anti-Luc siRNA-1 | 5′-GAU UAG GUC CGG UUA UGU AUU | 42 |
| Native siRNA-1 | UUCUA AUA CAG GCC AAU ACA Up-5′ | 43 |
| Anti-Luc siRNA-2 | 5′-CUG AAU ACA AAU CAC AGA AUU | 44 |
| Native siRNA-2 | UUGAC UUA UGU UUA GUG UCU UP-5′ | 45 |
| Anti-Luc siRNA-3 | 5′-UCC GGA AGC GAC CAA CGC CUU | 46 |
| Native siRNA-3 | UUAGG CCU UCG CUG GUU GCG GP-5′ | 47 |
| Non-specific Luc control siRNA | 5′-AUG UAU UGG CCU GUA UUA GUU | 48 |
| Control native siRNA | UUUAC AUA ACC GGA CAU AAU CP-5′ | 49 |
| Thiophosphate siRNA | | |
| duplexes | | |
| Thioluc-1 | 5′-CUG AAU ACA AAU CAC AGA AUU | 50 |
| thio siRNA-1 | UUGAC UUA UGU UUA GUG UCU UP-5′ | 45 |
| Thioluc-2 | 5′-CUG AAU ACA AAU CAC AGA AUU | 51 |
| thio siRNA-2 | UUGAC UUA UGU UUA GUG UCU UP-5′ | 45 |
| Thioluc-3 | 5′-CUG AAU ACA AAU CAC AGA AUU | 52 |
| thio siRNA-3 | UUGAC UUA UGU UUA GUG UCU UP-5′ | 45 |
| Thioluc-4 | 5′-CUG AAU AGA AAU CAC AGA AUU | 53 |
| thio siRNA-4 | UUGAC UUA UGU UUA GUG UCU UP-5′ | 45 |
| Thioluc-5 | 5′-CUG AAU AGA AAU CAC AGA AUU | 54 |
| thio siRNA-5 | UUGAC UUA UGU UUA GUG UCU UP-5′ | 45 |
| Thioluc-6 | 5′-CUG AAU ACA AAU CAC AGA AUU | 55 |
| thio siRNA-6 | UUGAC UUA UGU UUA GUG UCU UP-5′ | 45 |
| Non-specific Luc control thio | 5′-AUG UAU U GGC CUG UAU UAGUU | 56 |
| siRNA | | |
| Non-specific Luc control thio | UUUAC AUA A CC G GAC AUA AUCP-5′ | 57 |
| siRNA | | |

Note
Bold letters with underline in thio siRNAs represent the nucleotides with sulphur modifications

Reagents: The following reagents are included in the Dharmacon siSTARTER Kit: pGL3 plasmid (pGL3-expression vector); 1× Universal buffer (20 mM KCL, 6 mM HEPES-pH 7.5, 0.2 mM MgCL₂); transfection reagent (TransIT-TKO): 2.5 uM/ul of non-liposomal polymer/lipid formation; RNase-free water; Promega Steady-Glo Luciferase Assay Buffer; Promega Steady-Glo Luciferase Assay Subtrate. Opti-MEM (GIBCO); Dulbeco's Modifiied Eagles Medium (DMEM) with L-glutamine, pyridoxine hydrochloride, high glucose and without sodium pyruvate (GIBCO). Additionally, the following was addes to the medium (500 ml/bottle) before use: 25 ml of 5% inactivated fetal bovine serum, 10.6 ml of 20 mM HEPES, 5 ml of 1.8 mM glutamine and 0.5 ml of 50.8 uM 2-mercaptoethaonol; 1×PBS (GIBCO).

Luciferase gene silencing study: siRNA gene silencing studies were conducted according to the instructions of the Dharmacon siSTARTER Kit. Briefly, approximately 24 hours prior to transfection, 200 μl of 1×10⁵ HeLa cells (ATCC CCL-2)/ml DMEM suspension were placed into a 96-well plate in triplicate for each condition and were cultured at 37° C. with a 5% CO₂ atmosphere. The medium from cells to be transfected in each well was carefully removed and the cells were carefully washed twice with 50 μl of 1×PBS. After that, 100 μl of transfection complex solution containing siRNA duplex/TransIT-TKO/plasmid dilution mixture/serum, reporter plasmid only reagent mixture/serum, and no plasmid reagent mixture/serum was added to each relative well of 96-well plate respectively. Final concentration of native siRNA or thiophosphate siRNA in each well was 10 nM. The cells in the transfection reagent complex solution were continuously cultured for 48 hours at standard incubation conditions (37° C., 5% CO₂). Finally, the growth media from the cells in each well were removed and the cells were washed twice with 50 μl of 1×PBS again. 100 μl of Steady-Glo mixture containing Steady-Glo buffer and Steady-Glo substrate was added to each well and incubated at RT for 5 minutes. The luciferase activities of the samples were measured using a FLx800 Luminometer.

Concentration-response. The following concentrations of thiophosphate siRNAs and native siRNA-2 were designed for this study: 0.005 nM, 0.025 nM, 0.125 nM, 0.625 nM, 3.125 nM, 15.625 nM, 78.125 nM and 390.625 nM. The method and the cell line were same as described above.

Cytotoxicity studies. Briefly, 200 μl of 1×10⁵ same passage HeLa cells were placed in 96-well plates in triplicate for each treatment at the same time and incubated at 37° C. with a 5% CO₂ atmosphere. Transfection time and concentration of thiophosphate siRNAs and native siRNA-2 used in this study were the same as described in the Luciferase gene silencing studies. The number of cells including living cells and dead cells were counted under microscopy at two time points, 12 h and 48 h after transfection, using the Trypan Blue exclusion method. The formula used to calculate percent HeLa cell viability is: % viability=(number of living cells/total cell number)×100.

Statistical analyses: Data from triplicate for each condition mentioned above were expressed in mean±SD and statistical comparisons were performed by the Student's t-test. * indicates a statistically significant difference (P<0.05).

FIG. 12 is a graph that shows Effects of native siRNAs on Luciferase Gene Silencing in HeLa cells. Statistical results (Student t-test): Native siRNA-1 vs Native siRNA-2: P<0.01; Native siRNA-1 vs Native siRNA-3: P<0.05; Native siRNA-2 vs Native siRNA-3: P<0.05. Luminesence units were normalized to plasmid alone controls, and which the following were used:

1. native siRNA-1

2. native siRNA-2

3. native siRNA-3

4. Control native siRNA

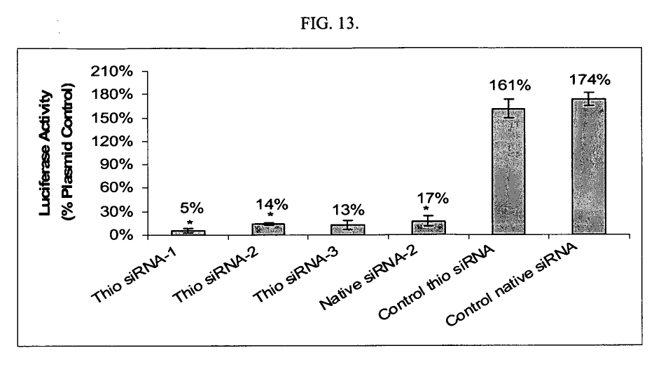

FIG. 13 is a graph that shows Effects of Thiophosphate siRNAs on Luciferase Gene Silencing in HeLa cells. Statistical results (Student t-test): Thio siRNA-1 vs Thio siRNA-2: P<0.05; Thio siRNA-1 vs Native siRNA-2: P<0.05. Luminesence units were normalized to plasmid alone controls, and which the following were used:

1. thio siRNA-1

2. thio siRNA-2

3. thio siRNA-3

4. native siRNA-2

5. Control thio siRNA

6. Control Native siRNA

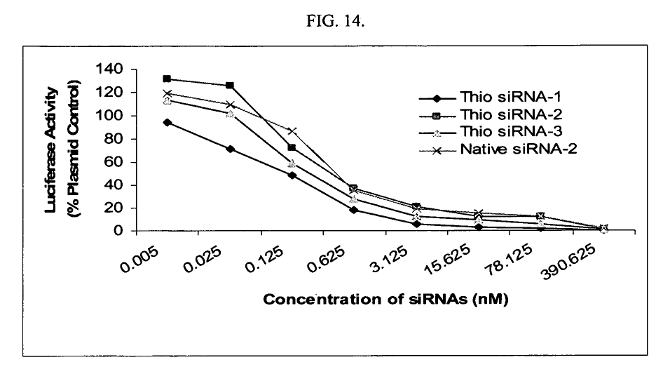

FIG. 14 is a Concentration-Response graph of Thiophosphate siRNAs Generated in HeLa Cells. The indicated concentrations of siRNAs were the final concentrations in the total transfection volume (0.1 ml). The plotted data are averages from triplicate±SD. Luminesence units were normalized to plasmid alone controls, and which the following were used:

1. Thio siRNA-1

2. Thio siRNA-2

3. Thio siRNA-3

4. Control Native siRNA Plasmid alone

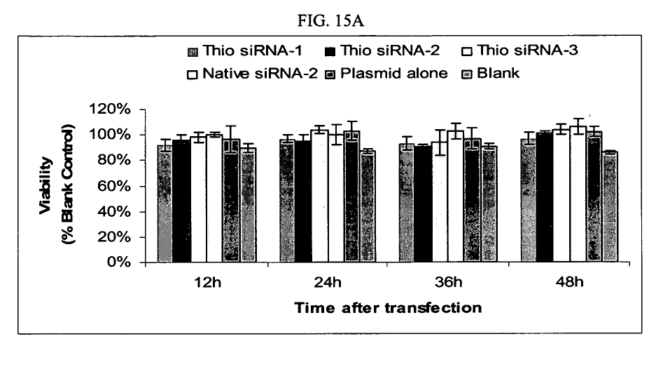

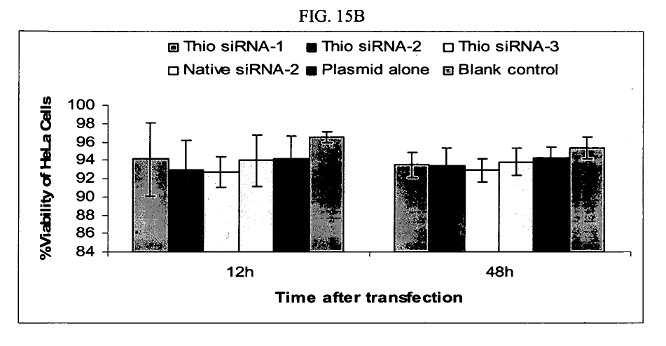

FIG. 15A is a graph of the effects of Thio siRNAs on HeLa cells Cytotoxicity. The HeLa cells were treated with 10 nM thiophosphate siRNAs and native siRNA-2 for 12 h, 24 h, 36 h and 48 hours.

1. thio siRNA-1

2. thio siRNA-2

3. thio siRNA-3

4. native siRNA-2

5. Plasmid alone

6. Blank

FIG. 15B is a graph of the effects of Thio siRNAs on HeLa cells Cytotoxicity. The HeLa cells were treated with 10 nM thiophosphate siRNAs and native siRNA-2 for 12 h and 48 hours.

1. thio siRNA-1

2. thio siRNA-2

3. thio siRNA-3

4. native siRNA-2

5. Plasmid alone

6. Blank

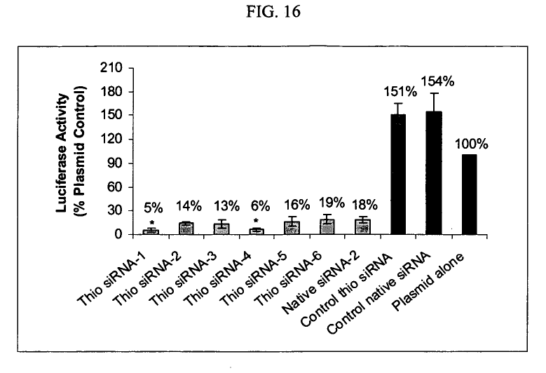

FIG. 16 is a graph of the effects of thiophosphate siRNAs on luciferase gene silencing in HeLa cells. Luminescence units were normalized to plasmid alone control.

1. thio siRNA-1

2. thio siRNA-2

3. thio siRNA-3

4. thio siRNA-4

5. thio siRNA-5

6. thio siRNA-6

7. native siRNA-2

8. Control thio siRNA

9. Control Native siRNA

10. Plasmid alone

FIG. 16 shows the base sequences from thioluc-1 through thioluc-6 are the same as anti-luc siRNA-2 and the sequence of control 1 is the same as non-specific luc control siRNA. The plasmid used was pGL3 plasmid (pGL3-expression vector) in a 1× Universal buffer: 20 mM KCl, 6 mM HEPES-pH 7.5, 0.2 mM MgCL₂. Transfection was accomplished using a TransIT-TKO transfection reagent: 2.5 μg/μl of non-liposomal polymer/lipid formulation. For detecting luciferase activity, the Promega Steady-Glo Luciferase Assay Buffer and Promega Steady-Glo Luciferase Assay Substrate kits were used according to the manufacturer's instructions. HeLa cells were grown in Opti-MEM (GIBCO) with 5% inactivated fetal bovine serum.

Cells were plated in 96-well plates in triplicate for each condition using 200 μl of 1×10⁵ HeLa cells/ml. Cells were incubated for 24 hours at 37° C. in 5% CO₂. siRNA or thio-modified siRNA duplexes and plasmid were resuspended as follows: 200 μl siRNA were resuspended in Universal Buffer to each siRNA tube for a final concentration of 1 μM. Next, 408 μl of Universal Buffer were added to each thioRNA tube for a concentration of 100 μM. For the thio-modified siRNAs, 2 ul of 100 μM thio siRNA duplex solution were added to 198 μl of the Universal Buffer to each tube for a final concentration of 1 μM. Finally, for the reporter plasmid, 40 μl RNase-free water were added to the 10 μg of reporter plasmid for a final concentration of 250 ng/μl.

As shown in FIG. 16 below, there was a statistically significant enhancement of silencing for the thiophosphate backbone substitution in thio-siRNA-1 and thio-siRNA-4 relative to the other sequences, with or without backbone substitutions, providing proof of principle that specific backbone substitutions in thio-modified siRNA can enhance activity.

FIG. 16. Effects of Thiophosphate siRNAs on Luciferase Gene Silencing in HeLa cells. Luminescence units were normalized to plasmid alone control. Statistical results: Thio siRNA-1 vs Thio siRNA-2, Thio siRNA-5, Thio siRNA-6 and Native siRNA-2 respectively: P<0.05; Thio siRNA-1 and Thio siRNA-4 vs Thio siRNA-5, Thio siRNA-6 and Native siRNA-2 respectively: P<0.05

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

M. Amarzguioui, et al., Nuc Acids Res, 31, 589-595, (2003)—Tolerance for mutations and chemical modifications in a siRNA.
D. A. Braasch, Y. Liu and D. R. Corey, Nucleic Acids Res, 30(23), 5160-7 (2002)—Antisense inhibition of gene expression in cells by oligonucleotides incorporating locked nucleic acids: effect of mRNA target sequence and chimera design
D. A. Braasch and D. R. Corey, Biochemistry, 41, 4503-4510 (2002)—Novel antisense and peptide nucleic acid strategies for controlling gene expression
D. A. Braasch, et al., Biochemistry, online release Jun. 11, 2003 (2003)—RNA interference in mammalian cells by chemically-modified RNA.
N. J. Caplen, et al., PNAS, 98, 9742-9747 (2001)—Specific inhibition of gene expression by small double-stranded RNAs in invertebrate and vertebrate systems.
J. T. Chi, PNAS, 100(11), 6343-6 (2003)—Genomewide view of gene silencing by small interfering RNAs.
S. Elbashir et al (Tuschi research group), 411, 494 (2001a)—Duplexes of 21-nt RNAs mediate RNA interference in cultured mammalian cells.
S. M. Elbashir, et al., EMBO J, 20, 6877-6888 (2001b)—Functional autonomy of siRNAs for mediating efficient RNAi in Drosophila melanogaster embryo lysate.
L. Gitlin, et al., Nature, 418, 430-434 (2002)—Short interfering RNA confers intracellular antiviral immunity in human cells.
D. E. Griffin, Alphaviruses. In “Fields' Virology, Fourth Edition,” D. M. Knipe, and P. M. Howley, editors, pp. 917-962. Lippincott, Williams and Wilkins, New York (2001). M. Hamada, et al., Antisense Nuc Acid Drug Dev, 12(5), 301-309 (2002)—Effects of RNA interference in gene expression (RNAi) in cultured mammalian cells of mismatches and the introduction of chemical modification at the 3′ ends of siRNAs.
W. Hu, et al., Curr Biol, 12, 1301-1311 (2002)—Inhibition of retroviral pathogenesis by RNA interference.
J. M. Jacque, et al., Nature, 418, 435-438 (2002)—Modulation of HIV-1 replication by RNA interference.
Fire, et al., Nature, 391, 806 (1998)—Potent and specific genetic interference by dsRNA in C. elegans
A. L. Jackson, et al., Nat Biotech, 21(6), 635-637 (2003)—Expression profiling reveals off-target gene regulation by RNAi.
B. Jansen and U. Zangemeister-Witte, Lancet Oncol, 3, 672-683 (2002)—Antisense therapy for cancer—the time of truth.
H. Kawasaki et al (Taira), Nuc Acids Res, 31(3), 981-987 (2003)—siRNAs generated by recombinant human Dicer include specific and significant but target site-independent gene silencing in human cells.
E. Koller, et al., Trends Pharm Sci, 21, 142-148—Elucidating cell signaling mechanisms using antisense technology.
N. S. Lee, et al., Nat Biotech, 19, 500-505 (2002)—Expression of small interfering RNAs targeted against HIV-1 rev transcripts in human cells.
J. Lescar, et al., Cell 105(1), 137-48. (2001)—The fusion glycoprotein shell of Semliki Forest virus: an icosahedral assembly primed for fusogenic activation at endosomal pH.
J. Matulic-Adamic, et al., J Org Chem., 61(11), 3909-3911 (1996)—Synthesis and Structure of 1-Deoxy-1-phenyl-beta-D-ribofuranose and Its Incorporation into Oligonucleotides.
V. M. Miller, et al., PNAS, 100(12), 7195-200—Allele-specific silencing of dominant disease genes.
M. Miyagishi et al (Taira), Antisense Nucleic Acid Drug Dev, 13(1), 1-7 (2003)—Comparison of the suppressive effects of antisense oligonucleotides and siRNAs against the same targets in mammalian cells.
C. D. Novina, et al., Nat Med, 8, 681-686 (2002)—siRNA-directed inibition of HIV-1 infection.
J. B. Opalinska, et al., Nature Rev Drug Discov, 1, 503-514 (2002)—Nucleic-acid therapeutics: basic principles and recent applications.
E. G. Marcusson, et al., Nuc Acids Res, 26, 2016-2023 (1998)—Phosphorothioate oligodeoxynucleotides dissociate from cationic lipids before entering the nucleus.
A. P. McCaffrey, et al., Nat Biotechnol, 21(6), 639-44 (2003)—Inhibition of hepatitis B virus in mice by RNA interference.
A. P. McCaffrey, et al., Nature, 418, 38-39 (2002)—RNA interference in adult mice.
S. Parrish et al (Fire research group), Mol Cell, 6, 1077-87 (2001)—Functional anatomy of a dsRNA trigger: differential requirement for the two trigger strands in RNA interference.
S. V. Pletnev, et al., Cell 105(1), 127-36 (2001)—Locations of carbohydrate sites on alphavirus glycoproteins show that E1 forms an icosahedral scaffold.
S. Schlesinger and M. J. Schlesinger, Togaviridae: The viruses and their replication. In “Fields' Virology, Fourth Edition” (D. M. Knipe, and P. M. Howley, Eds.), pp. 895-916. Lippincott, Williams and Wilkins, New York (2001).
D. Semizarov, et al., PNAS, 100(11), 6347-52 (2003)—Specificity of short interfering RNA determined through gene expression signatures.
E. Song, et al., Nat Med, 9, 347-351 (2003)—RNA interference targeting Fas protects mice from fulminant hepatitis.
E. Song, et al., J Virol. 2003 July;77(13):7174-81 (2003)—Sustained small interfering RNA-mediated Human Immunodeficiency Virus Type 1 inhibition in primary macrophages.
J. H. Strauss, J. H and E. G. Strauss, Microbiol. Rev. 58(3), 491-562 (1994)—The alphaviruses: gene expression, replication, and evolution.
F. Wianny and M. Zernicka-Goetz, Nat Cell Biol, 2, 70-75 (2000)—Novel antisense and peptide nucleic acid strategies for controlling gene expression.
H. B. Xia, et al., Nat Biotech, 20, 1006-1010 (2002)—siRNA-mediated gene silencing in vitro and in vivo.
T. Yokota et al (Taira), EMBO Rep., 4(6), 602-608 (2003)—Inhibition of intracellular hepatitis C virus by synthetic and vector-derived small interfering RNAs.
P. D. Zamore, Science, 296, 1265 (2002)—Ancient pathways programmed by small RNAs
Maniatis, et al., MOLECULAR CLONING: A LABORATORY MANUAL, 2nd Ed., Cold Spring Harbor (1989).

What is claimed is:

1. An isolated thioaptamer that mediates gene silencing, wherein the thioaptamer comprises a double-stranded hybrid thioaptamer.

2. The thioaptamer of claim 1, wherein the double-stranded hybrid thioaptamer is selected from RNA:DNA, RNA:RNA, DNA;DNA, PNA:DNA or PNA:RNA.

3. The thioaptamer of claim 1, wherein the double-stranded hybrid thioaptamer is thioated at one or more locations.

4. The thioaptamer of claim 1, wherein the double-stranded hybrid thioaptamer is dithioated.

5. The thioaptamer of claim 1, further comprising a terminal 3′ hydroxyl group.

6. The thioaptamer of claim 1, wherein the double-stranded hybrid thioaptamer comprises a perfect complementarity match to a target gene and gene silencing occurs by mRNA cleavage.

7. The thioaptamer of claim 1, further comprising a double-stranded hybrid thioaptamer having an imperfect complementarity match to a target gene and gene silencing occurs by mRNA cleavage.

8. The thioaptamer of claim 1, further comprising a double-stranded hybrid thioaptamer having an perfect complementarity match to a target gene and gene silencing occurs by repressed translation of mRNA to protein.

9. The thioaptamer of claim 1, wherein the thioaptamer comprises a double-stranded hybrid thioaptamer with an imperfect complementarity match to a target gene and gene silencing occurs by repressed translation of mRNA to protein.

10. The thioaptamer of claim 1, wherein one strand of the double-stranded hybrid thioaptamer comprises one or more of the following: rATP(αS), rUTP(αS), rGTP(αS), rCTP(αS), rATP(αS₂), rUTP(αS₂), rGTP(αS₂) or rCTP(αS₂).

11. The thioaptamer of claim 1, wherein one strand of the double-stranded hybrid thioaptamer comprises one or more of the following: dATP(αS), dTTP(αS), dGTP(αS), dCTP(αS), dATP(αS₂), dTTP(αS₂), dGTP(αS₂), or dTTP(αS₂).

12. An isolated thioaptamer selected from the group consisting of SEQ ID NO.: 26 to SEQ ID NO.: 57.

13. An isolated thioaptamer that mediates gene silencing, wherein the thioaptamer comprises a combination of short interfering DNA (siDNA); a micro, interfering DNA (miDNA); a small, temporal DNA (stDNA); a short, hairpin DNA (shDNA); short interfering RNA (siRNA); a micro, interfering RNA (mRNA); a small, temporal RNA (stRNA); or a short, hairpin RNA (shRNA).

14. The thioaptamer of claim 13, wherein the thioaptamer is monothioated.

15. The thioaptamer of claim 13, wherein the thioaptamer is thioated at one or more locations.

16. The thioaptamer of claim 13, further comprising a terminal hydroxyl group.

17. The thioaptamer of claim 13, wherein the thioaptamer comprises one or more of the following: dATP(αS), dTTP(αS), dGTP(αS), dCTP(αS), dATP(αS₂), dTTP(αS₂), dGTP(αS₂), dTTP(αS₂), rATP(αS), rUTP(αS), rGTP(αS), rCTP(αS), rATP(αS₂), rUTP(αS₂), rGTP(αS₂) or rCTP(αS₂).

18. A method of mediating gene silencing of a target gene in a cell or organism comprising the steps of:
introducing a double-stranded hybrid thioaptamer into the cell or organism; and
maintaining the cell or organism under conditions in which gene silencing occurs, thereby mediating expression of the target gene in the cell or organism.

19. The method of claim 18, wherein the double-stranded hybrid thioaptamer is selected from RNA:DNA, RNA:RNA, DNA;DNA, PNA:DNA or PNA:RNA.

20. The method of claim 18, wherein the target gene is selected from a viral gene, a cellular gene, a fungal gene, an extrachromosomal gene, a chromosomal gene, a genomic gene, a messenger RNA, or a RNAi.

21. The method of claim 18, wherein gene silencing is defined further as degradation of an mRNA transcript of the target gene that is cleaved in the presence of the thioaptamer before it can express a protein.

22. The method of claim 18, wherein gene silencing is defined further as regulation of translation of the target gene when the thioaptamer binds an mRNA transcript of the target gene at or about its 3′ UTR.

23. A method of examining the function of a gene in a cell or organism comprising the steps of:
introducing a double-stranded hybrid thioaptamer that targets an mRNA of the gene for gene silencing into the cell or organism, thereby producing a test cell or test organism;
maintaining the test cell or test organism under conditions under which gene silencing of mRNA of the gene occurs, thereby producing a test cell or test organism in which mRNA of the gene is silenced; and
observing the phenotype of the test cell or test organism against an appropriate control cell or control organism to provide information about the function of the gene.

24. The method of claim 23, wherein the double-stranded hybrid thioaptamer is selected from RNA:DNA, RNA:RNA, DNA;DNA, PNA:DNA or PNA:RNA.

25. A method of assessing whether a gene product is a suitable target for drug discovery comprising the steps of:
introducing an double-stranded hybrid thioaptamer that mediates gene silencing into a cell or organism under conditions in which gene silencing of an mRNA for the target gene results in decreased expression of the gene; and
determining the effect of the decreased expression of the gene on the cell or organism, wherein if decreased expression has an effect, then the gene product is a target for drug discovery.

26. The method of claim 25, wherein the double-stranded hybrid thioaptamer is selected from RNA:DNA, RNA:RNA, DNA;DNA, PNA:DNA or PNA:RNA.

27. A pharmaceutical composition comprising a double-stranded hybrid thioaptamer that mediates thioaptamer gene silencing and an appropriate carrier.

28. A method for reducing the expression of a gene in a cell, comprising the steps of:
selecting a double-stranded hybrid thioaptamer that mediates gene silencing of the gene to which it corresponds; and
introducing the thioaptamer into the cell, wherein the thioaptamer mediates RNA interference of a targeted sequence.

29. The method of claim 28, wherein the double-stranded hybrid thioaptamer is selected from RNA:DNA, RNA:RNA, DNA;DNA, PNA:DNA or PNA:RNA.

30. The method of claim 28, wherein the thioaptamer comprises double-stranded hybrid thioaptamer with a perfect complementarity match to a target gene and gene silencing occurs by mRNA cleavage.

31. The method of claim 28, wherein the thioaptamer comprises double-stranded hybrid thioaptamer with a imperfect complementarity match to a target gene and gene silencing occurs by mRNA cleavage.

32. A method for attenuating expression of a target gene in cultured cells, comprising the step of:
introducing a double-stranded hybrid thioaptamer into the cells in an amount sufficient to attenuate expression of the target gene, wherein the double-stranded hybrid thioaptamer comprises a nucleotide sequence that hybridizes under stringent conditions to a nucleotide sequence of the target gene and mediates attenuation of protein expression for a gene to which it corresponds.

33. The method of claim 32, wherein the cell is in cell culture, a virus, a mammalian cell, a human cell or a stem cell.

34. A method of producing a double-stranded hybrid thioaptamer comprising the steps of:
combining a double-stranded hybrid thioaptamer precursor with a soluble extract that mediates gene silencing, thereby producing a precursor-extract mixture; and
maintaining the precursor-extract mixture under conditions in which the double-stranded hybrid thioaptamer is processed to the mature thioaptamer.

35. The method of claim 34, wherein the double-stranded hybrid thioaptamer is selected from RNA:DNA, RNA:RNA, DNA;DNA, PNA:DNA or PNA:RNA.

36. The method of claim 34, further comprising isolating the double-stranded hybrid thioaptamer from the precursor-extract mixture.

37. The method of claim 34, further comprising the steps of:
determining the sequence of the double-stranded hybrid thioaptamer and the location of one or more thio-modifications to the thioaptamer; and
chemically synthesizing the thioaptamer.

38. A thioaptamer produced by the method of claim 34.

39. A method of mediating gene silencing of a target gene in a cell or organism comprising the steps of:
introducing a thioaptamer into the cell or organism, wherein the thioaptamer comprises a combination of short interfering DNA (siDNA); a micro, interfering DNA (miDNA); a small, temporal DNA (stDNA); a short, hairpin DNA (shDNA); short interfering RNA (siRNA); a micro, interfering RNA (mRNA); a small, temporal RNA (stRNA); or a short, hairpin RNA (shRNA); and
maintaining the cell or organism under conditions in which gene silencing occurs, thereby mediating expression of the target gene in the cell or organism.

40. The method of claim 39, wherein the cell is in cell culture, a virus, a mammalian cell, a human cell or a stem cell.

41. The method of claim 39, wherein gene silencing is defined further as degradation of an mRNA transcript of the target gene that is cleaved in the presence of the thioaptamer before it can express a protein.

42. The method of claim 39, wherein gene silencing is defined further as regulation of translation of the target gene when the thioaptamer binds an mRNA transcript of the target gene at or about its 3′UTR.

43. The thioaptamer of claim 39, wherein the thioaptamer comprises a combination of short interfering DNA (siDNA); a micro, interfering DNA (miDNA); a small, temporal DNA (stDNA); a short, hairpin DNA (shDNA); short interfering RNA (siRNA); a micro, interfering RNA (mRNA); a small, temporal RNA (stRNA); or a short, hairpin RNA (shRNA) with a perfect complementarity match to a target gene and gene silencing occurs by mRNA cleavage.

44. The thioaptamer of claim 39, wherein the thioaptamer comprises a combination of short interfering DNA (siDNA); a micro, interfering DNA (miDNA); a small, temporal DNA (stDNA); a short, hairpin DNA (shDNA); short interfering RNA (siRNA); a micro, interfering RNA (mRNA); a small, temporal RNA (stRNA); or a short, hairpin RNA (shRNA) with an imperfect complementarity match to a target gene and gene silencing occurs by repressed translation of mRNA to protein.

45. A method of examining the function of a gene in a cell or organism comprising the steps of:
introducing a thioaptamer comprises a combination of short interfering DNA (siDNA); a micro, interfering DNA (miDNA); a small, temporal DNA (stDNA); a short, hairpin DNA (shDNA); short interfering RNA (siRNA); a micro, interfering RNA (mRNA); a small, temporal RNA (stRNA); or a short, hairpin RNA (shRNA) that targets an mRNA of the gene for gene silencing into the cell or organism, thereby producing a test cell or test organism;
maintaining the test cell or test organism under conditions under which gene silencing of mRNA of the gene occurs, thereby producing a test cell or test organism in which mRNA of the gene is silenced; and
observing the phenotype of the test cell or test organism against an appropriate control cell or control organism to provide information about the function of the gene.

46. A method of assessing whether a gene product is a suitable target for drug discovery comprising the steps of:
introducing a thioaptamer comprises a combination of short interfering DNA (siDNA); a micro, interfering DNA (miDNA); a small, temporal DNA (stDNA); a short, hairpin DNA (shDNA); short interfering RNA (siRNA); a micro, interfering RNA (mRNA); a small, temporal RNA (stRNA); or a short, hairpin RNA (shRNA) that mediates gene silencing into a cell or organism under conditions in which gene silencing of an mRNA for the target gene results in decreased expression of the gene; and
determining the effect of the decreased expression of the gene on the cell or organism, wherein if decreased expression has an effect, then the gene product is a target for drug discovery.

47. A pharmaceutical composition comprising a thioaptamer that mediates thioaptamer gene silencing and an appropriate carrier, wherein the thioaptamer comprises a combination of short interfering DNA (siDNA); a micro, interfering DNA (miDNA); a small, temporal DNA (stDNA); a short, hairpin DNA (shDNA); short interfering RNA (siRNA); a micro, interfering RNA (mRNA); a small, temporal RNA (stRNA); or a short, hairpin RNA (shRNA).

48. A method for reducing the expression of a gene in a cell, comprising the steps of:
selecting a thioaptamer comprises a combination of short interfering DNA (siDNA); a micro, interfering DNA (miDNA); a small, temporal DNA (stDNA); a short, hairpin DNA (shDNA); short interfering RNA (siRNA); a micro, interfering RNA (mRNA); a small, temporal RNA (stRNA); or a short, hairpin RNA (shRNA) that mediates gene silencing of the gene to which it corresponds; and
introducing the thioaptamer into the cell, wherein the thioaptamer mediates RNA interference of a targeted sequence.

49. The method of claim 48, wherein the thioaptamer comprises an imperfect complementarity match to a target gene and gene silencing occurs by mRNA cleavage.

50. The method of claim 48, wherein the thioaptamer comprises a perfect complementarity match to a target gene and gene silencing occurs by mRNA cleavage.

51. A method for attenuating expression of a target gene in cultured cells, comprising the step of:
introducing a thioaptamer comprises a combination of short interfering DNA (siDNA); a micro, interfering DNA (miDNA); a small, temporal DNA (stDNA); a short, hairpin DNA (shDNA); short interfering RNA (siRNA); a micro, interfering RNA (mRNA); a small, temporal RNA (stRNA); or a short, hairpin RNA (shRNA) into the cells in an amount sufficient to attenuate expression of the target gene, wherein the thioaptamer comprises a nucleotide sequence that hybridizes under stringent conditions to a nucleotide sequence of the target gene and mediates attenuation of protein expression for a gene to which it corresponds.

52. The method of claim 51, wherein the cell is in cell culture, culture, a virus, a mammalian cell, a human cell or a stem cell.

53. A method of producing a thioaptamer comprising the steps of:
combining a combination of short interfering DNA (siDNA); a micro, interfering DNA (miDNA); a small, temporal DNA (stDNA); a short, hairpin DNA (shDNA); short interfering RNA (siRNA); a micro, interfering RNA (mRNA); a small, temporal RNA (stRNA); or a short, hairpin RNA (shRNA) thioaptamer precursor with a soluble extract that mediates gene silencing, thereby producing a precursor-extract mixture; and
maintaining the precursor-extract mixture under conditions in which a combination of short interfering DNA (siDNA); a micro, interfering DNA (miDNA); a small, temporal DNA (stDNA); a short, hairpin DNA (shDNA); short interfering RNA (siRNA); a micro, interfering RNA (mRNA); a small, temporal RNA (stRNA); or a short, hairpin RNA (shRNA) thioaptamer is processed to the mature thioaptamer.

54. The method of claim 53, further comprising isolating the thioaptamer from the precursor-extract mixture.

55. The method of claim 53, further comprising the step of determining the sequence of the thioaptamer and the location of one or more thio-modifications to the mature thioaptamer.

56. A thioaptamer produced by the method of claim 53.

57. The thioaptamer of claim 56, comprising SEQ ID NO.: 26 to SEQ ID NO.: 57.


SEQUENCE LISTING (57 sequences)

SEQ ID NO: 1 - RNA, 30 nt, Artificial
  Aptamer
cugccuacgccaugcccagaacccucacgc

SEQ ID NO: 2 - RNA, 30 nt, Artificial
  Aptamer
ggcccuugcgcccacacgcaaacaccgccc

SEQ ID NO: 3 - RNA, 30 nt, Artificial
  Aptamer
cgccaaccgaccgucccgaccguccgccuc

SEQ ID NO: 4 - RNA, 30 nt, Artificial
  Aptamer
ugcccaggccgcggccaucacuacuacgcc

SEQ ID NO: 5 - RNA, 30 nt, Artificial
  Aptamer
gcucagauccccccgccccgcuauccgcac

SEQ ID NO: 6 - RNA, 28 nt, Artificial
  Aptamer
uccugugcccggacccuguccccugcug

SEQ ID NO: 7 - RNA, 30 nt, Artificial
  Aptamer
ugccaagucggcuuccauccaccacccgag

SEQ ID NO: 8 - RNA, 30 nt, Artificial
  Aptamer
ccgacggaauucccgcuaguuccccugacc

SEQ ID NO: 9 - RNA, 28 nt, Artificial
  Apatmer
ccgacgacugauuauuccccugccccca

SEQ ID NO: 10 - RNA, 27 nt, Artificial
  Aptamer
ucacaccacacgcuucauccccugcac

SEQ ID NO: 11 - RNA, 29 nt, Artificial
  Aptamer
accucacaacuucgcaccucaaccgucuc

SEQ ID NO: 12 - RNA, 30 nt, Artificial
  Aptamer
cccucagcacuugcuugccagcggacgcca

SEQ ID NO: 13 - RNA, 30 nt, Artificial
  Aptamer
auggcuuacaagccgcagcucuauguggac

SEQ ID NO: 14 - RNA, 77 nt, Artificial
  Aptamer
gggagcucagaauaaacgcucaaggcccuugcgcccacacgcaaacaccgcccuucgacaugaggcccggauccggc

SEQ ID NO: 15 - RNA, 46 nt, Artificial
  Aptamer
gggagcucagaauaaacgcucaaggcccuugcgcccacacgcaagc

SEQ ID NO: 16 - RNA, 44 nt, Artificial
  Aptamer
ggcccuugcgcccacacgcaaacaccgcccgcccggauccggcc

SEQ ID NO: 17 - RNA, 45 nt, Artificial
  Aptamer
gguugcgcccacacgcaaacaccgcccuucgacaugaggcccggc

SEQ ID NO: 18 - RNA, 79 nt, Artificial
  Aptamer
gggagcucagaauaaacgcucaaggcccuugcgcccacacgcaaacaccgcccuucgacaugaggcccggauccggcuu

SEQ ID NO: 19 - RNA, 79 nt, Artificial
  Aptamer
gggagcucagaauaaacgcucaacugccuacgccaugcccagaacccucacgguucgacaugaggcccggauccggcuc

SEQ ID NO: 20 - RNA, 79 nt, Artificial
  Aptamer
gggagcucagaauaaacgcucaacugccuacgccaugcccagaacccucacgcuucgacaugaggcccggauccggcuu

SEQ ID NO: 21 - RNA, 61 nt, Artificial
  Aptamer
gggagcucagaauaaacgcucaaugcccauccugcuucgacaugaggcccggauccggcuu

SEQ ID NO: 22 - DNA, 15 bp, Artificial
  aptamer
ggatccggtggtctg

SEQ ID NO: 23 - DNA, 15 bp, Artificial
  Aptamer
cctactcgcgaattc

SEQ ID NO: 24 - RNA, 31 nt, Artificial
  Aptamer
gauccugaaacuguuuuaagguuggccgauc

SEQ ID NO: 25 - RNA, 31 nt, Artificial
  Aptamer
cuaggacuuggcacaaccgucacacugcuau

SEQ ID NO: 26 - RNA, 21 nt, Artificial
  Aptamer
gauuauguccgguuauguauu

SEQ ID NO: 27 - RNA, 21 nt, Artificial
  Aptamer
uacauaaccggacauaaucuu

SEQ ID NO: 28 - RNA, 21 nt, Artificial
  Aptamer
cugaauacaaaucacagaauu

SEQ ID NO: 29 - RNA, 21 nt, Artificial
  Aptamer
uucugugauuuguauucaguu

SEQ ID NO: 30 - RNA, 21 nt, Artificial
  Aptamer
uccggaagcgaccaacgccuu

SEQ ID NO: 31 - RNA, 21 nt, Artificial
  Aptamer
ggcguuggucgcuuccggauu

SEQ ID NO: 32 - RNA, 21 nt, Artificial
  Aptamer
auguauuggccuguauuaguu

SEQ ID NO: 33 - RNA, 21 nt, Artificial
  Aptamer
cuaauacaggccaauacauuu

SEQ ID NO: 34 - RNA, 21 nt, Artificial
  Aptamer
cugaauacaaaucacagaauu

SEQ ID NO: 35 - RNA, 21 nt, Artificial
  Aptamer
uucugugauuuguauucaguu

SEQ ID NO: 36 - RNA, 21 nt, Artificial
  Aptamer
cugaauacaaaucacagaauu

SEQ ID NO: 37 - RNA, 21 nt, Artificial
  Aptamer
cugaauacaaaucacagaauu

SEQ ID NO: 38 - RNA, 21 nt, Artificial
  Aptamer
cugaauacaaaucacagaauu

SEQ ID NO: 39 - RNA, 21 nt, Artificial
  Aptamer
cugaauacaaaucacagaauu

SEQ ID NO: 40 - RNA, 21 nt, Artificial
  Aptamer
cugaauacaaaucacagaauu

SEQ ID NO: 41 - RNA, 21 nt, Artificial
  Aptamer
auguauuggccuguauuaguu

SEQ ID NO: 42 - RNA, 21 nt, Artificial
  Aptamer
gauuagguccgguuauguauu

SEQ ID NO: 43 - RNA, 21 nt, Artificial
  Aptamer
uacauaaccggacauaaucuu

SEQ ID NO: 44 - RNA, 21 nt, Artificial
  Aptamer
cugaauacaaaucacagaauu

SEQ ID NO: 45 - RNA, 21 nt, Artificial
  Aptamer
uucugugauuuguauucaguu

SEQ ID NO: 46 - RNA, 21 nt, Artificial
  Aptamer
uccggaagcgaccaacgccuu

SEQ ID NO: 47 - RNA, 21 nt, Artificial
  Aptamer
ggcguuggucgcuuccggauu

SEQ ID NO: 48 - RNA, 21 nt, Artificial
  Aptamer
auguauuggccuguauuaguu

SEQ ID NO: 49 - RNA, 21 nt, Artificial
  Aptamer
cuaauacaggccaauacauuu

SEQ ID NO: 50 - RNA, 21 nt, Artificial
  Aptamer
cugaauacaaaucacagaauu

SEQ ID NO: 51 - RNA, 21 nt, Artificial
  Aptamer
cugaauacaaaucacagaauu

SEQ ID NO: 52 - RNA, 21 nt, Artificial
  Aptamer
cugaauacaaaucacagaauu

SEQ ID NO: 53 - RNA, 21 nt, Artificial
  Aptamer
cugaauacaaaucacagaauu

SEQ ID NO: 54 - RNA, 21 nt, Artificial
  Aptamer
cugaauacaaaucacagaauu

SEQ ID NO: 55 - RNA, 21 nt, Artificial
  Aptamer
cugaauacaaaucacagaauu

SEQ ID NO: 56 - RNA, 21 nt, Artificial
  Aptamer
auguauuggccuguauuaguu

SEQ ID NO: 57 - RNA, 21 nt, Artificial
  Aptamer
cuaauacaggccaauacauuu